(19) United States
(12) Patent Application Publication
(10) Pub. No.: US 2026/0200983 A1
(43) Pub. Date: Jul. 16, 2026

(54) USE OF GALR2 AGONISTS TO TREAT GASTROINTESTINAL AND/OR ENDOCRINE DISORDERS

(71) Applicant: NEURACLE SCIENCE CO., LTD., Seoul (KR)

(72) Inventors: Jae Young SEONG, Seoul (KR); Soon-gu KWON, Seoul (KR); Dong Sik KIM, Seoul (KR); Nui HA, Anyang-si (KR); Eun-Ho CHO, Seoul (KR); Hoyun KWAK, Hanam-si (KR); Wonkyum KIM, Sejong-si (KR); Hyun-Sook JANG, Seoul (KR)

(73) Assignee: NEURACLE SCIENCE CO., LTD., Seoul (KR)

(21) Appl. No.: 19/133,423

(22) PCT Filed: Nov. 29, 2023
(86) PCT No.: PCT/IB2023/062037

§ 371 Date: May 28, 2025

Related U.S. Application Data
(60) Provisional application No. 63/385,363, filed on Nov. 29, 2022.

(51) Int. Cl. C07K 7/08; A61K 9/00; A61K 38/00; A61K 45/06; A61K 47/60; A61P 1/10

(52) U.S. Cl. CPC C07K 7/08; A61K 9/0019; A61K 9/0043; A61K 45/06; A61K 47/60; A61P 1/10; A61K 38/00

(57) ABSTRACT

The present disclosure provides agonists against galanin receptor type 2 (GALR2 agonist) and the use of such agonists to treat gastrointestinal and/or endocrine disorders. Compared to other GALR2 ligands (e.g., wild-type spexin and/or galanin), the GALR2 agonists described herein differ (both functionally and/or structurally) such that they are more effective in treating the disorders described herein.

### CROSS-REFERENCE TO RELATED APPLICATION

This PCT application claims the priority benefit of U.S. Provisional Application No. 63/385,363, filed on Nov. 29, 2022, which is herein incorporated by reference in its entirety.

### REFERENCE TO SEQUENCE LISTING SUBMITTED ELECTRONICALLY

The content of the sequence listing is submitted electronically (Name: 3763_021PC01_Seqlisting_ST26. xml; Size: 108,231 bytes; and Date of Creation: Nov. 27, 2023) with the application and herein incorporated by reference in its entirety.

### FIELD OF THE DISCLOSURE

The present disclosure provides agonists specific for galanin receptor type 2 (GALR2) and their uses to treat a wide ranges of diseases, such as gastrointestinal and/or endocrine disorders.

### BACKGROUND OF THE DISCLOSURE

Diseases and disorders of the gastrointestinal and/or endocrine system continue to be a major health problem worldwide. For instance, among those suffering from a central nervous system injury or disease, bowel symptoms are extremely common. Emmanuel, A., F1000Research 8 (F1000 Faculty Rev): 1800 (2019). In fact, among many spinal cord injury patients, bowel dysfunction is reported as being much more problematic than any of bladder dysfunction, sexual dysfunction, pain, fatique, or perception of body image. Such symptoms are quite common in many endocrine disorders. Maser et al., World J Gastroenterol 12 (20): 3174-3179 (2006). Accordingly, there remains a need for alternative treatments for gastrointestinal and/or endocrine disorders that are safe and effective.

### BRIEF SUMMARY OF THE DISCLOSURE

Provided herein is a method of treating a gastrointestinal disorder in a subject in need thereof comprising administering to the subject a galanin receptor type 2 (GALR2) agonist, a nucleic acid encoding the GALR2 agonist, or a vector comprising the nucleic acid, wherein the GALR2 agonist comprises the amino acid sequence set forth in
X¹WX³X⁴X⁵X⁶X⁷X⁸YLX¹¹X¹²X¹³X¹⁴ (SEQ ID NO: 1), wherein:
X¹ is asparagine (N), glycine (G), pyroglutamate (pQ), or citrulline (Cit);
X³ is threonine (T), alanine (A), or lysine (K);
X⁴ is proline (P), leucine (L), glutamate (E), arginine (R), alanine (A), or valine (V);
X⁵ is asparagine (N) or glutamine (Q);
X⁶ is alanine (A) or serine(S);
X⁷ is alanine (A) or methionine (M);
X⁸ is leucine (L), glutamine (Q), or glycine (G);
X¹¹ is leucine (L), phenylalanine (F), tyrosine (Y), or aspartic acid (D);
X¹² is glycine (G) or alanine (A);
X¹³ is proline (P), arginine (R), or alanine (A);
X¹⁴ is glutamine (Q), histidine (H), or valine (V); and
wherein the GALR2 agonist specifically activates GALR2.

In some aspects, the gastrointestinal disorder comprises a constipation, neurogenic bowel dysfunction (NBD), or both. In some aspects, the constipation comprises an opioid-induced constipation (OIC).

The present disclosure further provides a method of regulating a bowel movement in a subject in need thereof comprising administering to the subject a galanin receptor type 2 (GALR2) agonist, a nucleic acid sequence encoding the GALR2 agonist, or a vector comprising the nucleic acid, wherein the GALR2 agonist comprises the amino acid sequence set forth in
X¹WX³X⁴X⁵X⁶X⁷X⁸YLX¹¹X¹²X¹³X¹⁴ (SEQ ID NO: 1), wherein:
X¹ is asparagine (N), glycine (G), pyroglutamate (pQ), or citrulline (Cit);
X³ is threonine (T), alanine (A), or lysine (K);
X⁴ is proline (P), leucine (L), glutamate (E), arginine (R), alanine (A), or valine (V);
X⁵ is asparagine (N) or glutamine (Q);
X⁶ is alanine (A) or serine(S);
X⁷ is alanine (A) or methionine (M);
X⁸ is leucine (L), glutamine (Q), or glycine (G);
X¹¹ is leucine (L), phenylalanine (F), tyrosine (Y), or aspartic acid (D);
X¹² is glycine (G) or alanine (A);
X¹³ is proline (P), arginine (R), or alanine (A);
X¹⁴ is glutamine (Q), histidine (H), or valine (V); and
wherein the GALR2 agonist specifically activates GALR2.

In some aspects, regulating a bowel movement comprises regulating a colonic transit time in the subject. In some aspects, after the administration of the GALR2 agonist, the colonic transit time in the subject is decreased compared to that of a reference subject (e.g., the subject prior to the administration and/or a corresponding subject who did not receive the administration). In some aspects, the colonic transit time is decreased by at least about 5%, at least about 10%, at least about 20%, at least about 30%, at least about 40%, at least about 50%, at least about 60%, at least about 70%, at least about 80%, at least about 90%, or about 100% as compared to that of the reference subject.

Also provided herein is a method of treating an endocrine disorder in a subject in need thereof comprising administering to the subject a galanin receptor type 2 (GALR2) agonist, a nucleic acid sequence encoding the GALR2 agonist, or a vector comprising the nucleic acid sequence, which comprises the amino acid sequence set forth in
X¹WX³X⁴X⁵X⁶X⁷X⁸YLX¹¹X¹²X¹³X¹⁴ (SEQ ID NO: 1), wherein:
X¹ is asparagine (N), glycine (G), pyroglutamate (pQ), or citrulline (Cit);
X³ is threonine (T), alanine (A), or lysine (K);
X⁴ is proline (P), leucine (L), glutamate (E), arginine (R), alanine (A), or valine (V);
X⁵ is asparagine (N) or glutamine (Q);
X⁶ is alanine (A) or serine(S);
X⁷ is alanine (A) or methionine (M);
X⁸ is leucine (L), glutamine (Q), or glycine (G);
X¹¹ is leucine (L), phenylalanine (F), tyrosine (Y), or aspartic acid (D);
X¹² is glycine (G) or alanine (A);
X¹³ is proline (P), arginine (R), or alanine (A);
X¹⁴ is glutamine (Q), histidine (H), or valine (V); and
wherein the GALR2 agonist specifically activates GALR2.

In some aspects, the endocrine disorder comprises a chronic renal failure, hypercalcemia, or both.

For any of the methods provided herein (e.g., provided above), in some aspects, the N at X¹ is D-asparagine. In some aspects, the W at position 2 of SEQ ID NO: 1 is D-tryptophan. In some aspects, the A at X⁴ is D-alanine, D-glutamate, or D-arginine. In some aspects, the V at X⁴ is D-valine. In some aspects, the A at X⁶ is D-alanine. In some aspects, the K at X¹¹ is D-lysine. In some aspects, the A at X¹² is D-alanine. In some aspects, the A at X¹³ is D-alanine. In some aspects, the Q at X¹⁴ is D-glutamine.

For any of the methods provided herein (e.g., provided above), in some aspects, the GALR2 agonist does not activate: (i) galanin receptor type 1 (GALR1), (ii) galanin receptor type 3 (GALR3), or (iii) both (i) and (ii).

For any of the methods provided herein (e.g., provided above), in some aspects, X⁷ is A and X¹¹ is F. In some aspects, X⁵ is N, X⁷ is A, and X¹¹ is F. In some aspects, X⁵ is N, X⁷ is A, X¹¹ is F, and X¹³ is P.

For any of the methods provided herein (e.g., provided above), in some aspects, the amino acid sequence of the GALR2 agonist comprises the sequence set forth in SEQ ID NO: 9, SEQ ID NO: 10, SEQ ID NO: 11, SEQ ID NO: 12, SEQ ID NO: 13, SEQ ID NO: 14, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 31, SEQ ID NO: 32, SEQ ID NO: 33, SEQ ID NO: 37, SEQ ID NO: 39, SEQ ID NO: 40, SEQ ID NO: 41, SEQ ID NO: 42, SEQ ID NO: 44, SEQ ID NO: 45, SEQ ID NO: 47, SEQ ID NO: 48, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 87, SEQ ID NO: 88, SEQ ID NO: 89, SEQ ID NO: 90, or SEQ ID NO: 91.

For any of the methods provided herein (e.g., provided above), in some aspects, the amino acid sequence of the GALR2 agonist is attached to a polyethylene glycol (PEG), an acetyl (Ac) group, or a Fmoc. In some aspects, X¹ is N, which is protected with the polyethylene glycol (PEG), acetyl (Ac) group, or Fmoc.

For any of the methods provided herein (e.g., provided above), in some aspects, the amino acid sequence of the GALR2 agonist is attached to the NH₂ on the C-terminus.

For any of the methods provided herein (e.g., provided above), in some aspects, the GALR2 agonist is administered to the subject intranasally, parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracistemally, intracapsularly, topically, orally, or combinations thereof. In some aspects, the GALR2 agonist is administered to the subject subcutaneously, intranasally, or intraperitoneally.

For any of the methods provided herein (e.g., provided above), in some aspects, the GALR2 agonist is administered to the subject one time, two times, three times, four times, five times, six times, or seven times or more.

For any of the methods provided herein (e.g., provided above), in some aspects, the methods further comprise administering an additional therapeutic agent to the subject. In some aspects, the additional therapeutic agent comprises a laxative (e.g., bisacodyl), opioid receptor antagonist (e.g., naloxone methiodide), irrigation (e.g., transanal or colonic), electrical stimulation, or combinations thereof. In some aspects, the additional therapeutic agent and the GALR2 agonist are administered to the subject concurrently. In some aspects, the additional therapeutic agent and the GALR2 agonist are administered to the subject sequentially. In some aspects, the GALR2 agonist is administered to the subject as a freeze-dried powder or solution.

### BRIEF DESCRIPTION OF THE DRAWINGS

FIG. 1 provides the overall experimental design in assessing the effects of GALR2 agonists described herein in normal naïve ICR mice. The upward arrows (seven total) represent the daily administration of the GALR2 agonist. Control animals received either a vehicle control (negative control) or bisacodyl (positive control). Glass bead was rectally inserted into the animals 30 minutes after the last treatment administration. Colonic transit time was assessed by measuring the latency to glass bead expulsion by the animals.

FIG. 2 shows the colonic transit time (in seconds) in normal naïve ICR mice that received a pegylated GALR2 (PEG-GALR2) agonist via intraperitoneal or intranasal administration. The treatment groups were as follows: (G1) vehicle control (intraperitoneally), (G2) PEG-GALR2 agonist (1 mg/kg; intraperitoneally), (G3) vehicle control (intranasally), (G4) PEG-GALR2 agonist (10 μg/head; intranasally), and (G5) bisacodyl (100 mg/kg; orally). The overall experimental design was as described in FIG. 1. Statistical analysis was performed using Student's t-test. “*”=a significant difference at p<0.05 level compared to the G3.

FIG. 3 shows the dose dependent effect of a pegylated-GALR2 (PEG-GALR2) agonist on colonic transit time in normal naïve ICR mice. The different treatment groups were as follows: (G1) vehicle control (intraperitoneally), (G2) 0.1 mg/kg of PEG-GALR2 agonist (intraperitoneally), (G3) 0.3 mg/kg of PEG-GALR2 agonist (intraperitoneally), (G4) 1 mg/kg of PEG-GALR2 agonist (intraperitoneally), (G5) 1 mg/kg of PEG-GALR2 agonist (subcutaneously), and (G6) bisacodyl (100 mg/kg; orally). The overall experimental design was as described in FIG. 1. Statistical analysis was performed using Student's t-test. “*”=a significant difference at p<0.05 level compared to G1.

FIG. 4 provides the overall experimental design in assessing the effects of GALR2 agonists described herein in an opioid-induced constipation (OIC) mouse model. The upward arrows (seven total) represent the daily administration of the GALR2 agonist. The GALR2 agonist that was administered to the relevant groups was pegylated. Control animals received either a vehicle control (negative control) or bisacodyl (positive control). Animals that received a single administration (i.e., at day 7) of naloxone methiodide were also used as a positive control. At 10 minutes post last administration, each of the animals received an administration of morphine (3 mg/kg; subcutaneously). Glass bead was rectally inserted into the animals 30 minutes after the last treatment administration. Colonic transit time was assessed by measuring the latency to glass bead expulsion by the animals.

FIG. 5 shows colonic transit time in OIC mice that received pegylated GALR2 (PEG-GALR2) agonist via different routes of administration (i.e., intraperitoneal, intranasal, or subcutaneous). The treatment groups were as follows: (G1) normal naïve mice (no OIC); (G2) vehicle control (intraperitoneally), (G3) PEG-GALR2 agonist (1 mg/kg; intraperitoneally), (G4) vehicle control (intranasally), (G5) PEG-GALR2 agonist (10 μg; intranasally), (G6) bisacodyl (100 mg/kg; orally) (positive control), and (G7) naloxone methiodide (10 mg/kg; intraperitoneally) (positive control). The overall experimental design was as described in FIG. 4. Statistical analysis was performed using Student's t-test. “***/**/*”=a significant difference at p<0.001/0.01/0.05 level, respectively, compared to G1. “###”=a significant difference at p<0.001 level compared to G2. “$$$”=a significant difference at p<0.001 level compared to G4.

FIG. 6 shows the dose dependent effect of GALR2 agonists on colonic transit time in OIC mice. The different treatment groups were as follows: (G1) normal naïve mice (no OIC), (G2) vehicle control (intraperitoneally), (G3) 0.1 mg/kg of PEG-GALR2 agonist (intraperitoneally), (G4) 0.3 mg/kg of PEG-GALR2 agonist (intraperitoneally), (G5) 1 mg/kg of PEG-GALR2 agonist (intraperitoneally), (G6) vehicle control (intranasally), (G7) 1 μg of PEG-GALR2 agonist (intranasally), (G8) 3 μg of PEG-GALR2 agonist (intranasally), (G9) 10 μg of PEG-GALR2 agonist (intranasally), (G10) bisacodyl (100 mg/kg; orally), and (G11) naloxone methiodide (10 mg/kg; intraperitoneally). The overall experimental design was as described in FIG. 4. Statistical analysis was performed using Student's t-test. “***/**”=a significant difference at p<0.001/0.01 level, respectively, compared to G1. “###/##/#”=a significant difference at p<0.001/0.01/0.05 level, respectively, compared to G2. “$$$/$$/$”=a significant difference at p<0.001/0.01/0.05 level, respectively, compared to G6.

FIG. 7 shows the effect of administration route (intraperitoneal vs. subcutaneous) on the dose dependent effect of GALR2 agonists on colonic transit time in OIC mice. The different treatment groups were as follows: (G1) normal naïve mice (no OIC), (G2) vehicle control (intraperitoneally), (G3) 0.5 mg/kg of PEG-GALR2 agonist (intraperitoneally), (G4) 1 mg/kg of PEG-GALR2 agonist (intraperitoneally), (G5) vehicle control (subcutaneously), (G6) 0.5 mg/kg of PEG-GALR2 agonist (subcutaneously), (G7) 1 mg/kg of PEG-GALR2 agonist (subcutaneously), (G8) bisacodyl (100 mg/kg; orally), and (G9) naloxone methiodide (10 mg/kg; intraperitoneally). The overall experimental design was as described in FIG. 4. Statistical analysis was performed using Student's t-test. “***/**”=a significant difference at p<0.001/0.01 level, respectively, compared to G1. “###”=a significant difference at p<0.001 level compared to G2. “$$$”=a significant difference at p<0.001 level compared to G5.

FIG. 8 provides a comparison of colonic transit time in OIC mice treated with either the wild-type spexin peptide or GALR2 agonists described herein via subcutaneous administration. The GALR2 agonist was pegylated (PEG-GALR2 agonist) or non-pegylated (GALR2 agonist). The different treatment groups were as follows: (G1) normal naïve mice (no OIC), (G2) vehicle control, (G3) 0.5 mg/kg of PEG-GALR2 agonist, (G4) 1 mg/kg of PEG-GALR2 agonist, (G5) 0.5 mg/kg of GALR2 agonist, (G6) 1 mg/kg of GALR2 agonist, (G7) 0.5 mg/kg of wild-type spexin, (G8) 1 mg/kg of wild-type spexin, (G9) bisacodyl (100 mg/kg; orally, and (G10) naloxone methiodide (10 mg/kg; intraperitoneally). The overall experimental design was as described in FIG. 4. “**/**/*”=a significant difference at p<0.001/0.01/0.05 level, respectively, compared to G1 (t-test). “###/##/#”=a significant difference at p<0.001/0.01/0.05 level, respectively, compared to G2 (t-test). “$”=a significant difference at p<0.05 level compared to G8 (G4, G6, G8, one-way ANOVA, Bonferroni's multiple comparison test).

FIGS. 9A and 9B show the effect of dosing schedule and administration route on GALR2 agonist-mediated regulation of colonic transit time in OIC mice. FIG. 9A provides a schematic of the overall experimental design. Non-pegylated GALR2 agonist was administered to the OIC mice as follows: (i) daily for seven days via subcutaneous administration (1 mg/kg per dose) (“G3”), (ii) single subcutaneous administration at day seven (“G4”) (1 mg/kg), (iii) single intranasal administration at day seven (“G5”) (10 μg/head). Some of the animals received a single administration of a Fc-conjugated non-pegylated GALR2 (GALR2-Fc) agonist on day four (35 mg/kg) (“G6”). Normal naïve mice (no OIC; “G1”) and OIC mice subcutaneously treated with a vehicle control (“G2”) were used as controls. At 10 minutes post last administration, each of the animals received an administration of morphine (3 mg/kg; subcutaneously). Glass bead was rectally inserted into the animals 30 minutes after the last treatment administration. Colonic transit time was assessed by measuring the latency to glass bead expulsion by the animals. FIG. 9B provides a comparison of colonic transit time among the different treatment groups. “***/**”=a significant difference at p<0.0001/0.01 level, respectively, compared to G1. “###”=a significant difference at p<0.001 level compared to G2.

FIGS. 10A, 10B, and 10C show the effect of GALR2 agonist on colonic transit time after multiple induction of opioid-mediated constipation. FIG. 10A shows the overall experimental design. Mice received a single administration of the GALR2 agonist either subcutaneously (1 mg/kg) (G3) or intranasally (10 μg/head) (G4). Normal naïve mice (no OIC) (G1) and OIC mice treated with a vehicle control (deionized water) (G2) were used as controls. For the first OIC induction, 4 hours after treatment administration, the relevant animals received a subcutaneous administration of morphine (3 mg/kg), and then 30 minutes later, glass bead was rectally inserted. Then, a first colonic transit time was assessed in the animals for 60 minutes. At 48 hours after treatment administration, second OIC was induced by a second subcutaneous administration of morphine (3 mg/kg), and then 30 minutes later, glass bead was again rectally inserted. Then, a second colonic transit time was assessed in the animals for 60 minutes. Colonic transit time was assessed by measuring the latency to glass bead expulsion by the animals. FIG. 10B provides a comparison of the first colonic transit time. FIG. 10C provides a comparison of the second colonic transit time. “***”=significant difference at p<0.001 compare to G1 (T-test). “###”=significant difference at p<0.001 compared to G2 (T-test).

FIG. 11 provides a comparison of the in vitro potency of three different GALR2 agonist peptides with certain amino acid substitutions at the 4th amino acid position. Specifically, the GALR2 agonist peptides were as follows: (1) nWTaNAALYLFGPq-NH₂ (D-alanine substitution; triangle), (2) PEG2-NWTeNAALYLFGPq-NH₂ (D-glutamic acid; filled circle), and (3) PEG2-NWTrNAALYLFGPq-NH₂ (D-arginine; open circle). Potency of the GALR2 agonists was assessed by measuring SRE luciferase activity in mGqi-hGALR2-SRE Luc expressing cell lines treated with the peptides.

### DETAILED DESCRIPTION OF THE DISCLOSURE

The present disclosure is generally directed to methods of treating various diseases and disorders (e.g., gastrointestinal, endocrine, and/or metabolic disorders) comprising administering to a subject an agonist of galanin receptor type 2 (“GALR2 agonist”). As further described herein, the GALR2 agonists of the present disclosure exhibit one or more properties such that they differ (e.g., structurally and/or functionally) from other GALR2 ligands (e.g., wild-type spexin). Additional aspects of the present disclosure are provided throughout the present application.

To facilitate an understanding of the disclosure disclosed herein, a number of terms and phrases are defined. Additional definitions are set forth throughout the detailed description.

### I. Definitions

Throughout this disclosure, the term “a” or “an” entity refers to one or more of that entity; for example, “an antibody,” is understood to represent one or more antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “galanin receptor type 2 agonist” or “GALR2 agonist” refers to any molecule that is capable of binding to GALR2 and thereby, activating the GALR2-mediated signaling pathway. As described herein, in some aspects, GALR2 agonists described herein do not activate GALR1 and GALR3 (i.e., specific to GALR2). And, unless indicated otherwise, GALR2 agonist of the present disclosure comprise one or more of the modifications (e.g., amino acid modifications) described herein. Non-limiting examples of such modifications are provided throughout the present disclosure.

The term “galanin receptor type 2” (GALR2) refers to a G-protein coupled galanin (GAL) receptor that is encoded by the GALR2 gene. In addition to GALR2, there are two other GAL receptor subtypes: galanin type receptor 1 (GALR1) and galanin type receptor 3 (GALR3). The human GALR1 gene contains three exons and is translated into a 349-aa protein (see Table 1). The homology between species is 93% for rat and human GALR1. The expression of GALR1 is regulated by cAMP through the transcription factor CREB. Human GALR2 has 92% sequence identity to rat GALR2, although there is a 15-aa extension of the C-terminal end in human GALR2. The amino acid sequence for human GALR2, which is 387 amino acids in length, is set forth in Table 1. The GALR2 gene is expressed more ubiquitously compared with that of GALR1, as it is found in several peripheral tissues, including the pituitary gland, gastrointestinal tract, skeletal muscle, heart, kidney, uterus, ovary, and testis, in addition to the central nervous system. Lastly, human GALR3 consists of 368 aa (see Table 1) and shares 36% identity with human GALR1, 58% with human GALR2, and approximately 90% with rat GALR3.

TABLE 1
Human GAL Receptor Sequences

| | |
|---|---|
| GALR1 | MELAVGNLSEGNASWPEPPAPEPGPLFGIGVEN |
| (UniProt: | FVTLVVFGLIFALGVLGNSLVITVLARSKPGKP |
| P47211-1) | RSTTNLFILNLSIADLAYLLFCIPFQATVYALP |
| (SEQ ID | TWVLGAFICKFIHYFFTVSMLVSIFTLAAMSVD |
| NO: 80) | RYVAIVHSRRSSSLRVSRNALLGVGCIWALSIA |
| | MASPVAYHQGLFHPRASNQTFCWEQWPDPRHKK |
| | AYVVCTFVFGYLLPLLLICFCYAKVLNHLHKKL |
| | KNMSKKSEASKKKTAQTVLVVVVVFGISWLPHH |
| | IIHLWAEFGVFPLTPASFLFRITAHCLAYSNSS |
| | VNPIIYAFLSENFRKAYKQVFKCHIRKDSHLSD |
| | TKESKSRIDTPPSTNCTHV |
| GALR2 | MNVSGCPGAGNASQAGGGGGWHPEAVIVPLLFA |
| (UniProt: | LIFLVGTVGNTLVLAVLLRGGQAVSTTNLFILN |
| O43603-1) | LGVADLCFILCCVPFQATIYTLDGWVFGSLLCK |
| (SEQ ID | AVHFLIFLTMHASSFTLAAVSLDRYLAIRYPLH |
| NO: 81) | SRELRTPRNALAAIGLIWGLSLLFSGPYLSYYR |
| | QSQLANLTVCHPAWSAPRRRAMDICTFVFSYLL |
| | PVLVLGLTYARTLRYLWRAVDPVAAGSGARRAK |
| | RKVTRMILIVAALFCLCWMPHHALILCVWFGQF |
| | PLTRATYALRILSHLVSYANSCVNPIVYALVSK |
| | HFRKGFRTICAGLLGRAPGRASGRVCAAARGTH |
| | SGSVLERESSDLLHMSEAAGALRPCPGASQPCI |
| | LEPCPGPSWQGPKAGDSILTVDVA |
| GALR3 | MADAQNISLDSPGSVGAVAVPVVFALIFLLGTV |
| (UniProt: | GNGLVLAVLLQPGPSAWQEPGSTTDLFILNLAV |
| O60755-1) | ADLCFILCCVPFQATIYTLDAWLFGALVCKAVH |
| (SEQ ID | LLIYLTMYASSFTLAAVSVDRYLAVRHPLRSRA |
| NO: 82) | LRTPRNARAAVGLVWLLAALFSAPYLSYYGTVR |
| | YGALELCVPAWEDARRRALDVATFAAGYLLPVA |
| | VVSLAYGRTLRFLWAAVGPAGAAAAEARRRATG |
| | RAGRAMLAVAALYALCWGPHHALILCFWYGRFA |
| | FSPATYACRLASHCLAYANSCLNPLVYALASRH |
| | FRARFRRLWPCGRRRRHRARRALRRVRPASSGP |
| | PGCPGDARPSGRLLAGGGQGPEPREGPVHGGEA |
| | ARGPE |

Natural ligands of the GAL receptors (including GALR2) are known and include: galanin and spexin. “Galanin” is an important neuromodulator that is widely distributed throughout the body (e.g., brain, gastrointestinal system, and hypothalamusry axis). Sipkova, J., et al., Physiol Res 66:729-740 (2017), which is incorporated herein by reference in its entirety. At least in humans, galanin is a 30-amino acid non-C-terminally amidated peptide that plays a role in many biological functions, such as somatosensory transmission, smooth muscle contractility, hormone release, and feeding. The amino acid sequence for the human galanin is provided in Table 2. The precursor peptide is 123 amino acids in length (SEQ ID NO: 83) and is proteolytically processed to produce the mature galanin peptide (SEQ ID NO: 84). More specifically, (i) amino acids 1-19 correspond to the signal peptide, (ii) amino acids 20-30 correspond to the propeptide, (iii) amino acids 33-62 correspond to the galanin peptide (SEQ ID NO: 84), and (iv) amino acids 65-123 correspond to the galanin message-associated peptide.

TABLE 2
Human Galanin Sequence

| | |
|---|---|
| Galanin Precursor | MARGSALLLASLLLAAALSASAGLWSPA |
| Peptide (UniProt: | KEKRGWTLNSAGYLLGPHAVGNHRSFSD |
| P22466-1) (SEQ ID | KNGLTSKRELRPEDDMKPGSFDRSIPEN |
| NO: 83) | NIMRTIIEFLSFLHLKEAGALDRLLDLP |
| | AAASSEDIERS |
| Galanin Mature | GWTLNSAGYLLGPHAVGNHRSFSDKNGL |
| Peptide (SEQ ID | TS |
| NO: 84) | |

“Spexin” (also known as NPQ, SPX, and neuropeptide Q) is a more recently discovered neuropeptide that shares many similarities to galanin. Like galanin, spexin is also distributed in various tissues and plays a role in many different biological functions (e.g., GI tract movement, energy balance and weight loss, fatty acid uptake, glucose homeostasis, nociception and cardiovascular/renal functions). However, unlike galanin which can activate all GALR subtypes, spexin is specific to GALR2 and GALR3 but not GALR1. The mature spexin peptide sequence consists of 14 amino acids (see Table 3) formed as a result of cleavage of dibasic amino acids by a proprotein convertase and is very well conserved in typical vertebrate species as well as humans.

TABLE 3
Spexin Sequence

| Human Spexin | MKGLRSLAATTLALFLVFVFLGNSSCAP |
|---|---|
| Precursor (UniProt: | QRLLERRNWTPQAMLYLKGAQGRRFISD |
| Q9BT56-1) (SEQ ID | QSRRKDLSDRPLPERRSPNPQLLTIPEA |
| NO: 85) | ATILLASLQKSPEDEEKNFDQTRFLEDS |
| | LLNW |
| Spexin Mature | NWTPQAMLYLKGAQ |
| Peptide (SEQ ID NO: | |
| 86) | |

As used herein, the term “gastrointestinal disorder” refers to any disease or disorder that affects the upper and/or lower gastrointestinal tract of a subject. Non-limiting examples of such disorders include: heartburn, inflammatory bowel disease, Crohn's disease, ulcerative colitis, peptic ulcers, stress ulcers, bleeding peptic ulcers, duodenal ulcers, infectious enteritis, colitis, diverticulitis, gastric hyperacidity, dyspepsia, gastroparesis, Zollinger-Ellison syndrome, gastroesophageal reflux disease (“GERD”) (i.e., acid reflux), including, but not limited to, symptomatic GERD and asymptomatic GERD, Helicobacter pylori associated-diseases, hypersecretory states associated with systemic mastocytosis or basophilic leukemia and hyperhistaminemia that result, for example, from neurosurgery, head injury, severe body trauma or burns. In some aspects, a gastrointestinal disorder does not comprise irritable bowel syndrome. As is apparent from the present disclosure, in some aspects the gastrointestinal disorder can be associated with abnormal nerve function. Unless indicated otherwise, such gastrointestinal disorders are also referred to herein as “neurogenic bowel dysfunction.” In some aspects, the gastrointestinal disorder that can be treated with the present disclosure is associated with impaired intestinal motility. Not to be bound by any one theory, in some aspects, a GALR2 agonist described herein can treat such a gastrointestinal disorder by promoting (e.g., increasing) intestinal motility.

As used herein, the term “lower gastrointestinal tract” refers to the ileum, the colon, the cecum, and/or the rectum. As used herein, the term “upper gastrointestinal tract” refers to the esophagus, the stomach, the duodenum, and/or the jejunum.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless indicated otherwise, the terms “polypeptide” and “protein” are used interchangeably herein.

The terms “nucleic acid molecule,” “nucleic acid,” and “polynucleotide,” as used herein, can be used interchangeably and is intended to include DNA molecules and RNA molecules. A nucleic acid molecule can be single-stranded or double-stranded, and can be cDNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, the term “linked” or “conjugated” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein, “administering” refers to the physical introduction of a therapeutic agent or a composition comprising a therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

As used herein, the term “subject” includes any human or non-human animal. The term “non-human animal” includes all vertebrates, e.g., mammals and non-mammals, such as non-human primates, sheep, dog, cow, chickens, amphibians, reptiles, etc.

The term “therapeutically effective amount” as used herein refers to an amount of a drug, alone or in combination with another therapeutic agent, effective to “treat” a disease or disorder in a subject or reduce the risk, potential, possibility or occurrence of a disease or disorder (e.g., a gastrointestinal and/or endocrine disorder). A “therapeutically effective amount” includes an amount of a drug or a therapeutic agent that provides some improvement or benefit to a subject having or at risk of having a disease or disorder (e.g., a gastrointestinal and/or endocrine disorder). Thus, a “therapeutically effective” amount is an amount that reduces the risk, potential, possibility or occurrence of a disease or provides disorder or some alleviation, mitigation, and/or reduces at least one indicator, and/or decrease in at least one clinical symptom of a disease or disorder.

### II. Methods of the Disclosure

Some aspects of the present disclosure is directed to methods of treating a disease or disorder in a subject in need thereof. Unless indicated otherwise, a disease or disorder that can be treated with the present disclosure does not comprise any of the following: attention deficit hyperactivity disorder (ADHD), bipolar disorder, body dysmorphic disorder, bulimia nervosa and other eating disorders, cataplexy, dysthymia, general anxiety disorder, hypersexuality, irritable bowel syndrome, impulse-control disorder (MDD), kleptomania, migraine, major depressive disorder, narcolepsy, obsessive-compulsive disorder, oppositional-defiant disorder, panic disorder, post-traumatic stress disorder (PTSD), premenstrual dysphoric disorder (PMDD), social anxiety disorder, chronic pain, intermittent explosive disorder, pathological gambling, personality disorder, pyromania, substance abuse and addiction, trichotillomania, Alzheimer's disease, or obesity disorder. In some aspects, a disease or disorder that can be treated with the present disclosure does not comprise attention deficit hyperactivity disorder (ADHD). In some aspects, a disease or disorder that can be treated with the present disclosure does not comprise bipolar disorder. In some aspects, a disease or disorder that can be treated with the present disclosure does not comprise body dysmorphic disorder. In some aspects, a disease or disorder that can be treated with the present disclosure does not comprise bulima. In some aspects, a disease or disorder that can be treated with the present disclosure does not comprise an eating disorder (e.g., nervosa). In some aspects, a disease or disorder that can be treated with the present disclosure does not comprise cataplexy. In some aspects, a disease or disorder that can be treated with the present disclosure does not comprise dysthymia. In some aspects, a disease or disorder that can be treated with the present disclosure does not comprise general anxiety disorder. In some aspects, a disease or disorder that can be treated with the present disclosure does not comprise hypersexuality. In some aspects, a disease or disorder that can be treated with the present disclosure does not comprise irritable bowel syndrome. In some aspects, a disease or disorder that can be treated with the present disclosure does not comprise impulse-control disorder (MDD). In some aspects, a disease or disorder that can be treated with the present disclosure does not comprise kleptomania. In some aspects, a disease or disorder that can be treated with the present disclosure does not comprise migraine. In some aspects, a disease or disorder that can be treated with the present disclosure does not comprise major depressive disorder. In some aspects, a disease or disorder that can be treated with the present disclosure does not comprise narcolepsy. In some aspects, a disease or disorder that can be treated with the present disclosure does not comprise obsessive-compulsive disorder. In some aspects, a disease or disorder that can be treated with the present disclosure does not comprise oppositional-defiant disorder. In some aspects, a disease or disorder that can be treated with the present disclosure does not comprise panic disorder. In some aspects, a disease or disorder that can be treated with the present disclosure does not comprise post-traumatic stress disorder (PTSD). In some aspects, a disease or disorder that can be treated with the present disclosure does not comprise premenstrual dysphoric disorder (PMDD). In some aspects, a disease or disorder that can be treated with the present disclosure does not comprise social anxiety disorder. In some aspects, a disease or disorder that can be treated with the present disclosure does not comprise chronic pain. In some aspects, a disease or disorder that can be treated with the present disclosure does not comprise intermittent explosive disorder. In some aspects, a disease or disorder that can be treated with the present disclosure does not comprise pathological gambling. In some aspects, a disease or disorder that can be treated with the present disclosure does not comprise personality disorder. In some aspects, a disease or disorder that can be treated with the present disclosure does not comprise pyromania. In some aspects, a disease or disorder that can be treated with the present disclosure does not comprise substance abuse and addiction. In some aspects, a disease or disorder that can be treated with the present disclosure does not comprise trichotillomania. In some aspects, a disease or disorder that can be treated with the present disclosure does not comprise Alzheimer's disease. In some aspects, a disease or disorder that can be treated with the present disclosure does not comprise obesity disorder.

### II.A. Gastrointestinal Disorders

In some aspects, a disease or disorder that can be treated with the present disclosure comprises a gastrointestinal disorders in a subject in need thereof, comprising administering to the subject any of the galanin receptor type 2 (GALR2) agonists described herein. As is apparent from the present disclosure, in some aspects, the GALR2 agonist is administered to the subject as a protein. In some aspects, the GALR2 agonist is administered to the subject as a nucleic acid (e.g., encoding any of the GALR2 agonists provided herein). In some aspects, administering a GALR2 agonist to a subject comprises administering a vector comprising a nucleic acid encoding any of the GALR2 agonists described herein. Accordingly, unless indicated otherwise, “administering a GALR2 agonist” comprises: (i) administering the GALR2 agonist itself to the subject (e.g., as a protein), (ii) administering a nucleic acid encoding a GALR2 agonist, (iii) administering a vector comprising a nucleic acid encoding a GALR2 agonist, and (iv) any combination of (i) to (iii).

In some aspects, the gastrointestinal disorders that can be treated with the present disclosure are associated with an abnormal nerve function. For instance, in some aspects, a gastrointestinal disorder exhibits impaired intestinal motility, wherein the impaired intestinal motility is associated with (e.g., caused by) the abnormal nerve function. Not to be bound by any one theory, in some aspects, the abnormal nerve function may lead to the inability to control one or more parts of the gastrointestinal tract (e.g., colon), resulting in the gastrointestinal disorder. As described herein, such gastrointestinal disorders are referred to herein as “neurogenic bowel dysfunction” or “NBD.” Accordingly, in some aspects, the present disclosure provides a method of treating a neurogenic bowel dysfunction in a subject in need thereof, comprising administering to the subject any of the GALR2 agonists described herein.

As is apparent from at least the above disclosure, the GALR2 agonists of the present disclosure can be used to treat a neurogenic bowel dysfunction associated with any type of nerve dysfunction. For instance, in some aspects, the NBD is associated with a physical injury (e.g., spinal cord injury). In some aspects, the NBD is associated with a neurological disease. Non-limiting examples of such neurological diseases include: multiple sclerosis (MS), amyotrophic lateral sclerosis (ALS), stroke, spina bifida, brain lesion, Parkinson's disease, diabetes mellitus, and combinations thereof. In some aspects, the NBD is associated with both a physical injury and a neurological disease.

As is also apparent from the present disclosure, in some aspects, a gastrointestinal disorder that can be treated with the present disclosure is not associated with an abnormal nerve cell function. For instance, in some aspects, a gastrointestinal disorder exhibits impaired intestinal motility, wherein the impaired intestinal motility is not associated with (e.g., caused by) the abnormal nerve cell function.

In some aspects, administering a GALR2 agonist described herein to a subject can help improve (e.g., ameliorate) one or more symptoms associated with the gastrointestinal disorder. For instance, in some aspects, after the administration of a GALR2 agonist described herein, one or more symptoms of NBD is improved in the subject. Non-limiting examples of such symptoms include: constipation, diarrhea, fecal incontinence. See, e.g., Emmanuel, A., F1000Research 8 (F1000 Faculty Rev): 1800 (2019), wherein is incorporated herein by reference in its entirety.

In some aspects, the methods provided herein are useful in treating a constipation in a subject in need thereof. In some aspects, such methods comprise administering any of the GALR2 agonists described herein to the subject, wherein after the administration, the constipation is improved (e.g., reduced) in the subject. As used herein, the term “constipation” refers to a physical condition that includes at least one of the following conditions: reduced frequency of bowel movements, hardening of feces, and difficulty in passing feces. Generally, a subject suffering from constipation can often suffer from straining during bowel movements and/or a sensation of incomplete evacuation following bowel movements. In some aspects, constipation refers to a subject who experiences less than three (3) rescue free bowel movements (RFBMs) per week on average, wherein “rescue free bowel movement” refers to the passage and evacuation of feces, or laxation. Accordingly, in some aspects, an improved constipation comprises: (i) a more frequent and regular bowel movement (e.g., three or more RFMBs per week on average), (ii) softening of feces, (iii) less difficulty in passing feces, or (iv) any combination thereof.

As is apparent from the present disclosure, the methods provided herein can be used to treat constipation associated with a wide range of causes. In some aspects, the constipation comprises an opioid-induced constipation. As used herein, “opioid-induced constipation” refers to any constipation resulting from the use of opioid drugs. As used herein, the term “opioid” refers to a compound that binds to opioid receptors. Unless indicated otherwise, the term “opioid drugs,” as used herein, comprises all natural and synthetic opioids. In some aspects, opioid drugs comprise drugs acting on opioid receptors present in the central nervous system and/or peripheral system, as well as those acting on opioid receptors present in the gastrointestinal tract. Non-limiting examples of natural opioids include: morphine, codeine, thebaine, and salvinorin A. Non-limiting examples of synthetic opioids include: semi-synthetic opium alkaloids derivatives such as heroin (diacetylmorphine), dihydrocodeine, hydromorphone, nicomorphine, and oxycodone. Illustrative examples of fully synthetic opioid drugs include, but are not limited to, anilidopiperidines (e.g., fentanyl), phenylpiperidines (e.g., pethidine), diphenylpropylamine derivatives (e.g., loperamide), benzomorphan derivatives (e.g., dezocine), oripavine derivatives (e.g., buprenorphine), and morphinan derivatives (e.g., butorphanol).

Not to be bound by any one theory, in some aspects, a GALR2 agonist described herein can treat a gastrointestinal disorder (e.g., neurogenic bowel dysfunction) by regulating a bowel movement in a subject. As used herein, the term “bowel movement” refers to the evacuation of feces from the gastrointestinal tract. For instance, where a subject is suffering from decreased bowel movement (e.g., constipation), in some aspects, administering a GALR2 agonist described herein can increase bowel movement in the subject. Accordingly, in some aspects, the present disclosure relating to a method of regulating a bowel movement in a subject suffering from a constipation, comprising administering to the subject any of the GALR2 agonists described herein, wherein after the administration, bowel movement in the subject is increased. In some aspects, the constipation comprises an opioid-induced constipation. As is apparent from the present disclosure, in some aspects, an increase in bowel movement refers to an increase in the number of rescue free bowel movement. In some aspects, bowel movement is increased in the subject by at least about 10%, at least about 20%, at least about 30%, at least about 40%, at least about 50%, at least about 60%, at least about 70%, at least about 80%, at least about 90%, or at least about 100%, as compared to that of a reference subject (e.g., corresponding subject who did not receive an administration of the GALR2 agonist and/or the subject prior to the administration of the GALR2 agonist). In some aspects, after the administration of the GALR2 agonist, the subject has three or more rescue free bowel movements per week on average.

Not to be bound by any one theory, in some aspects, a gastrointestinal disorder (e.g., neurogenic bowel dysfunction, e.g., opioid-induced constipation) can be treated by reducing colonic transit time in a subject. As used herein, the term “colonic transit time” refers to the amount of time it takes for a substance to move through the colon. As is apparent from the present disclosure, in some aspects, reduced colonic transit time can allow for greater gut motility and more frequent bowel movement. In some aspects, after the administration of a GALR2 agonist described herein, colonic transit time is decreased in the subject by at least about 5%, at least about 10%, at least about 20%, at least about 30%, at least about 40%, at least about 50%, at least about 60%, at least about 70%, at least about 80%, at least about 90%, or at least about 100%, as compared to that of a reference subject (e.g., corresponding subject who did not receive an administration of the GALR2 agonist and/or the subject prior to the administration). Colonic transit time can be assessed using any suitable methods known in the art (e.g., using radiopaque markers, radioactive isotopes, wireless motility capsule). Non-limiting example of such methods are described herein (see, e.g., Example 2).

### II.B. Endocrine Disorder

Some aspects of the present disclosure is directed to methods of treating an endocrine disorder in a subject in need thereof, comprising administering to the subject any of the GALR2 agonists described herein. Some aspects of the present disclosure is directed to methods of treating a metabolic disorder in a subject in need thereof, comprising administering to the subject any of the GALR2 agonists described herein. Some aspects of the present disclosure is directed to methods of treating both endocrine and metabolic disorders in a subject in need thereof, comprising administering to the subject any of the GALR2 agonists described herein. As used herein, “endocrine disorders” refer to diseases or conditions of the endocrine system. “Endocrine system” refers to the various glands and organs that make and release hormones that regulate various aspects of the body, including, but not limited to, growth and development, metabolism, and reproduction. In some aspects, the endocrine disorder comprises (i) endocrine gland hypofunction/hyposecretion (leading to hormone deficiency); (ii) endocrine glad hyperfunction/hypersecretion (leading to hormone excess); (iii) tumors (benign or malignant) of endocrine glands; or (iv) any combination of (i) to (iii).

Accordingly, in some aspects, the GALR2 agonists described herein can be useful in treating any endocrine disorders known in the art. Non-limiting examples of endocrine disorders include: glucose homeostasis disorder (e.g., diabetes, hypoglycemia, and glucagonoma), thyroid disorder (e.g., goiter, hyperthyroidism, hypothyroidism, thyroiditis, thyroid cancer, thyroid hormone resistance), calcium homeostasis disorders and metabolic bone diseases (e.g., hypercalcemia, parathyroid gland disorder, osteoporosis, osteitis deformans, rickets, and osteomalacia), posterior pituitary disorder (e.g., diabetes insipidus, syndrome of inappropriate antidiuretic hormone (SIADH)), anterior pituitary disorder (e.g., hypopituitarism and pituitary tumors), sex hormone disorder (e.g., disorders of sex development or intersex disorders, hypogonadism, disorders of puberty, menstrual function or fertility disorder), kidney disorder (e.g., chronic renal failure), multiple endocrine neoplasia, carcinoid syndrome, and combinations thereof. In some aspects, an endocrine disorder that can be treated using the methods provided herein comprises both chronic renal failure and hypercalcemia. In some aspects, the endocrine disorder is chronic renal failure. In some aspects, the endocrine disorder is hypercalcemia.

In any of the treatment methods provided herein, a GALR2 agonist described herein can be administered to a subject (e.g., suffering from a gastrointestinal disorder and/or an endocrine disorder) via any suitable routes. Non-limiting examples of such routes of administration include parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracistemally, intracapsularly, topically, or combinations thereof. In some aspects, a GALR2 agonist described herein is administered to a subject subcutaneously. In some aspects, a GALR2 agonist is administered to a subject intranasally. In some aspects, a GALR2 agonist described is administered to a subject intraperitoneally.

As demonstrated herein, in some aspects, a GALR2 agonist described herein can be administered to a subject multiple times. For instance, in some aspects, a GALR2 agonist is administered to a subject at least about two times, at least about three times, at least about four times, at least about five times, at least about six times, at least about seven times, at least about eight times, at least about nine times, or at least about 10 times. In some aspects, a GALR2 agonist is administered to the subject daily for seven consecutive days. Also, as demonstrated herein, in some aspects, a GALR2 agonist described herein can be administered to a subject one time. In some aspects, the GALR2 agonist described herein is administered to a subject at a dose of about 0.01 mg/kg to about 100 mg/kg. In some aspects, the GALR2 agonist described herein is administered to a subject at a dose of about 0.05 mg/kg to about 0.5 mg/kg. In some aspects, the GALR2 agonist is administered to a subject at a dose of about 0.05 mg/kg. In some aspects, the GALR2 agonist is administered to a subject at a dose of about 0.1 mg/kg. In some aspects, the GALR2 agonist is administered to a subject at a dose of about 0.2 mg/kg. In some aspects, the GALR2 agonist is administered to a subject at a dose of about 0.3 mg/kg. In some aspects, the GALR2 agonist is administered to a subject at a dose of about 0.4 mg/kg. In some aspects, the GALR2 agonist is administered to a subject at a dose of about 0.5 mg/kg. In some aspects, the GALR2 agonist is administered to a subject at a dose of about 0.6 mg/kg. In some aspects, the GALR2 agonist is administered to a subject at a dose of about 0.7 mg/kg. In some aspects, the GALR2 agonist is administered to a subject at a dose of about 0.8 mg/kg. In some aspects, the GALR2 agonist is administered to a subject at a dose of about 0.9 mg/kg. In some aspects, the GALR2 agonist is administered to a subject at a dose of about 1 mg/kg.

In some aspects, a treatment method described herein (e.g., to treat a gastrointestinal disorder and/or endocrine disorder) comprises administering to a subject in need thereof a GALR2 agonist and an additional therapeutic agent. In some aspects, the GALR2 agonist and the additional therapeutic agent can be administered to the subject concurrently. For instance, in some aspects, the GALR2 agonist and the additional therapeutic agent can be administered to the subject as a single composition, e.g., a pharmaceutical composition comprising both the GALR2 agonist and the additional therapeutic agent. In some aspects, the GALR2 agonist and the additional therapeutic agent are administered to the subject concurrently but as separate compositions, e.g., a first pharmaceutical composition comprising the GALR2 agonist and a second pharmaceutical composition comprising the additional therapeutic agent, wherein the first and second pharmaceutical compositions are administered to the subject concurrently. In some aspects, the GALR2 agonist and the additional therapeutic agent are administered to the subject sequentially. For instance, in some aspects, the GALR2 agonist is administered to the subject before the additional therapeutic agent. In some aspects, the GALR2 agonist is administered to the subject after the additional therapeutic agent.

As is apparent from the present disclosure, the additional therapeutic agent can comprise any treatment known in the art suitable for treating an indication described herein (e.g., gastrointestinal disorders and/or endocrine disorders). For instance, where a subject suffers from a gastrointestinal disorder (e.g., constipation), in some aspects, an additional therapeutic agent that can be administered to the subject in combination with a GALR2 agonist include a laxative (e.g., bisacodyl), opioid receptor antagonist (e.g., naloxone methiodide), irrigation (e.g., transanal or colonic), electrical stimulation, or combinations thereof.

### III. GALR2 Agonists

As described elsewhere in the present disclosure, GALR2 agonists useful for the present disclosure exhibits one or more properties such that they differ (e.g., structurally and/or functionally) from other GALR2 ligands. Non-limiting examples of such differences are described below. Additional disclosures of useful GALR2 agonists are provided, e.g., in U.S. Pat. No. 11,248,023, which is incorporated herein by reference in its entirety.

### III.A. Amino Acid Modifications

In some aspects, GALR2 agonists of the present disclosure comprise one or more amino acid modifications (e.g., substitutions, deletions, additions, and/or indels) as compared a reference GALR2 ligand (e.g., wild-type spexin and/or galanin). For instance, in some aspects, a GALR2 agonist useful for the present disclosure (e.g., can be used to treat a gastrointestinal disorder, endocrine disorder, or both) comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E) (also referred to herein as “glutamic acid”), arginine (R), alanine (A), or valine (V); (4) X⁵ is asparagine (N) or glutamine (Q); (5) X⁶ is alanine (A) or serine(S); (6) X⁷ is alanine (A) or methionine (M); (7) X⁸ is leucine (L), glutamine (Q), or glycine (G); (8) X¹¹ is leucine (L), phenylalanine (F), tyrosine (Y), or aspartic acid (D); (9) X¹² is glycine (G) or alanine (A); (10) X¹³ is proline (P), arginine (R), or alanine (A); (11) X¹⁴ is glutamine (Q), histidine (H), or valine (V); or (12) any combination of (1) to (11).

Accordingly, in some aspects, provided herein is a method of treating a gastrointestinal disorder in a subject in need thereof, comprising administering to the subject a GALR2 agonist, which comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E), arginine (R), alanine (A), or valine (V); (4) X⁵ is asparagine (N) or glutamine (Q); (5) X⁶ is alanine (A) or serine(S); (6) X⁷ is alanine (A) or methionine (M); (7) X⁸ is leucine (L), glutamine (Q), or glycine (G); (8) X¹¹ is leucine (L), phenylalanine (F), tyrosine (Y), or aspartic acid (D); (9) X¹² is glycine (G) or alanine (A); (10) X¹³ is proline (P), arginine (R), or alanine (A); (11) X¹⁴ is glutamine (Q), histidine (H), or valine (V); or (12) any combination of (1) to (11). In some aspects, provided herein is a method of regulating a bowel movement in a subject in need thereof, comprising administering to the subject a GALR2 agonist, which comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E), arginine (R), alanine (A), or valine (V); (4) X⁵ is asparagine (N) or glutamine (Q); (5) X⁶ is alanine (A) or serine(S); (6) X⁷ is alanine (A) or methionine (M); (7) X⁸ is leucine (L), glutamine (Q), or glycine (G); (8) X¹¹ is leucine (L), phenylalanine (F), tyrosine (Y), or aspartic acid (D); (9) X¹² is glycine (G) or alanine (A); (10) X¹³ is proline (P), arginine (R), or alanine (A); (11) X¹⁴ is glutamine (Q), histidine (H), or valine (V); or (12) any combination of (1) to (11). In some aspects, provided herein is a method of treating an endocrine disorder in a subject in need thereof, comprising administering to the subject a GALR2 agonist, which comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E), arginine (R), alanine (A), or valine (V); (4) X⁵ is asparagine (N) or glutamine (Q); (5) X⁶ is alanine (A) or serine(S); (6) X⁷ is alanine (A) or methionine (M); (7) X⁸ is leucine (L), glutamine (Q), or glycine (G); (8) X¹¹ is leucine (L), phenylalanine (F), tyrosine (Y), or aspartic acid (D); (9) X¹² is glycine (G) or alanine (A); (10) X¹³ is proline (P), arginine (R), or alanine (A); (11) X¹⁴ is glutamine (Q), histidine (H), or valine (V); or (12) any combination of (1) to (11).

In some aspects, the GALR2 agonist comprises the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E), arginine (R), alanine (A), or valine (V); (4) X⁵ is asparagine (N) or glutamine (Q); (5) X⁶ is alanine (A) or serine(S); (6) X⁷ is alanine (A) or methionine (M); (7) X⁸ is leucine (L), glutamine (Q), or glycine (G); (8) X¹¹ is leucine (L), phenylalanine (F), tyrosine (Y), or aspartic acid (D); (9) X¹² is glycine (G) or alanine (A); (10) X¹³ is proline (P), arginine (R), or alanine (A); (11) X¹⁴ is glutamine (Q), histidine (H), or valine (V); or (12) any combination of (1) to (11). In some aspects, the GALR2 agonist consists of the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E), arginine (R), alanine (A), or valine (V); (4) X⁵ is asparagine (N) or glutamine (Q); (5) X⁶ is alanine (A) or serine(S); (6) X⁷ is alanine (A) or methionine (M); (7) X⁸ is leucine (L), glutamine (Q), or glycine (G); (8) X¹¹ is leucine (L), phenylalanine (F), tyrosine (Y), or aspartic acid (D); (9) X¹² is glycine (G) or alanine (A); (10) X¹³ is proline (P), arginine (R), or alanine (A); (11) X¹⁴ is glutamine (Q), histidine (H), or valine (V); or (12) any combination of (1) to (11). In some aspects, the GALR2 agonist consists essentially of the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E), arginine (R), alanine (A), or valine (V); (4) X⁵ is asparagine (N) or glutamine (Q); (5) X⁶ is alanine (A) or serine(S); (6) X⁷ is alanine (A) or methionine (M); (7) X⁸ is leucine (L), glutamine (Q), or glycine (G); (8) X¹¹ is leucine (L), phenylalanine (F), tyrosine (Y), or aspartic acid (D); (9) X¹² is glycine (G) or alanine (A); (10) X¹³ is proline (P), arginine (R), or alanine (A); (11) X¹⁴ is glutamine (Q), histidine (H), or valine (V); or (12) any combination of (1) to (11).

In some aspects, X¹ is asparagine (N), glycine (G), pyroglutamate (pQ), or citrulline (Cit). In some aspects, X³ is threonine (T), alanine (A), or lysine (K). In some aspects, X⁴ is proline (P), leucine (L), glutamate (E), arginine (R), alanine (A), or valine (V). In some aspects, X⁵ is asparagine (N) or glutamine (Q). In some aspects, X⁶ is alanine (A) or serine(S). In some aspects, X⁷ is alanine (A) or methionine (M). In some aspects, X⁸ is leucine (L), glutamine (Q), or glycine (G). In some aspects, X¹¹ is leucine (L), phenylalanine (F), tyrosine (Y), or aspartic acid (D). In some aspects, X¹² is glycine (G) or alanine (A). In some aspects, X¹³ is proline (P), arginine (R), or alanine (A). In some aspects, X¹⁴ is glutamine (Q), histidine (H), or valine (V).

Unless indicated otherwise, the amino acids at each of the positions can be in the D-configuration or in the L-configuration. For instance, in some aspects, X¹ is L-asparagine or D-asparagine. In some aspects, X¹ is L-asparagine. In some aspects, X¹ is D-asparagine. In some aspects, X¹ is L-glycine or D-glycine. In some aspects, X¹ is L-glycine. In some aspects, X¹ is D-glycine. In some aspects, the W at position 2 of SEQ ID NO: 1 is L-tryptophan or D-tryptophan. In some aspects, the W at position 2 of SEQ ID NO: 1 is L-tryptophan. In some aspects, the W at position 2 of SEQ ID NO: 1 is D-tryptophan. In some aspects, X³ is L-threonine or D-threonine. In some aspects, X³ is L-threonine. In some aspects, X³ is D-threonine. In some aspects, X³ is L-alanine or D-alanine. In some aspects, X³ is L-alanine. In some aspects, X³ is D-alanine. In some aspects, X³ is L-lysine or D-lysine. In some aspects, X³ is L-lysine. In some aspects, X³ is D-lysine. In some aspects, X⁴ is L-proline or D-proline. In some aspects, X⁴ is L-proline. In some aspects, X⁴ is D-proline. In some aspects, X⁴ is L-leucine or D-leucine. In some aspects, X⁴ is L-leucine. In some aspects, X⁴ is D-leucine. In some aspects, X⁴ is L-glutamate or D-glutamate. In some aspects, X⁴ is L-glutamate. In some aspects, X⁴ is D-glutamate. In some aspects, X⁴ is L-arginine or D-arginine. In some aspects, X⁴ is L-arginine. In some aspects, X⁴ is D-arginine. In some aspects, X⁴ is L-valine or D-valine. In some aspects, X⁴ is L-valine. In some aspects, X⁴ is D-valine. In some aspects, X⁵ is L-asparagine or D-asparagine. In some aspects, X⁵ is L-asparagine. In some aspects, X⁵ is D-asparagine. In some aspects, X⁵ is L-glutamine or D-glutamine. In some aspects, X⁵ is L-glutamine. In some aspects, X⁵ is D-glutamine. In some aspects, X⁶ is L-alanine or D-alanine. In some aspects, X⁶ is L-alanine. In some aspects, X⁶ is D-alanine. In some aspects, X⁶ is L-serine or D-serine. In some aspects, X⁶ is L-serine. In some aspects, X⁶ is D-serine. In some aspects, X⁷ is L-alanine or D-alanine. In some aspects, X⁷ is L-alanine. In some aspects, X⁷ is D-alanine. In some aspects, X⁷ is L-methionine or D-methionine. In some aspects, X⁷ is L-methionine. In some aspects, X⁷ is D-methionine. In some aspects, X⁸ is L-leucine or D-leucine. In some aspects, X⁸ is L-leucine. In some aspects, X⁸ is D-leucine. In some aspects, X⁸ is L-glutamine or D-glutamine. In some aspects, X⁸ is L-glutamine. In some aspects, X⁸ is D-glutamine. In some aspects, X⁸ is L-glycine or D-glycine. In some aspects, X⁸ is L-glycine. In some aspects, X⁸ is D-glycine. In some aspects, the Y at position 9 of SEQ ID NO: 1 is L-tyrosine or D-tyrosine. In some aspects, the Y at position 9 of SEQ ID NO: 1 is L-tyrosine. In some aspects, the Y at position 9 of SEQ ID NO: 1 is D-tyrosine. In some aspects, the L at position 10 of SEQ ID NO: 1 is L-leucine or D-leucine. In some aspects, the L at position 10 of SEQ ID NO: 1 is L-leucine. In some aspects, the L at position 10 of SEQ ID NO: 1 is D-leucine. In some aspects, X¹¹ is L-leucine or D-leucine. In some aspects, X¹¹ is L-leucine. In some aspects, X¹¹ is D-leucine. In some aspects, X¹¹ is L-phenylalanine or D-phenylalanine. In some aspects, X¹¹ is L-phenylalanine. In some aspects, X¹¹ is D-phenylalanine. In some aspects, X¹¹ is L-tyrosine or D-tyrosine. In some aspects, X¹¹ is L-tyrosine. In some aspects, X¹¹ is D-tyrosine. In some aspects, X¹¹ is L-aspartic acid or D-aspartic acid. In some aspects, X¹¹ is L-aspartic acid. In some aspects, X¹¹ is D-aspartic acid. In some aspects, X¹² is L-glycine or D-glycine. In some aspects, X¹² is L-glycine. In some aspects, X¹² is D-glycine. In some aspects, X¹² is L-alanine or D-alanine. In some aspects, X¹² is L-alanine. In some aspects, X¹² is D-alanine. In some aspects, X¹³ is L-proline or D-proline. In some aspects, X¹³ is L-proline. In some aspects, X¹³ is D-proline. In some aspects, X¹³ is L-arginine or D-arginine. In some aspects, X¹³ is L-arginine. In some aspects, X¹³ is D-arginine. In some aspects, X¹³ is L-alanine or D-alanine. In some aspects, X¹³ is L-alanine. In some aspects, X¹³ is D-alanine. In some aspects, X¹⁴ is L-glutamine or D-glutamine. In some aspects, X¹⁴ is L-glutamine. In some aspects, X¹⁴ is D-glutamine. In some aspects, X¹⁴ is L-histidine or D-histidine. In some aspects, X¹⁴ is L-histidine. In some aspects, X¹⁴ is D-histidine. In some aspects, X¹⁴ is L-valine or D-valine. In some aspects, X¹⁴ is L-valine. In some aspects, X¹⁴ is D-valine.

Accordingly, in some aspects, a GALR2 agonist useful for the methods provided herein comprises, consists of, or consists essentially of an amino acid sequence comprising one or more D-amino acids. Not to be bound by any one theory, in some aspects, modifying a GALR2 agonist described herein to comprise one or more D-amino acids can enhance the persistence of the GALR2 agonists, e.g., when administered to a subject. For instance, the inclusion of the D-amino acids can protect the polypeptide from protease and peptidase degradation within the blood of the subject. Therefore, in some aspects, a GALR2 agonist described herein comprises, consists of, or consists essentially of an amino acid sequence comprising one or more D-amino acids, wherein the GALR2 agonist is more resistant to protease and/or peptidase degradation as compared to a reference GALR2 ligand. Unless indicated otherwise, the reference GALR2 ligand can comprise: (i) a wild-type galanin, (ii) a wild-type spexin, (iii) a corresponding GALR2 agonist without the one or more D-amino acids, or (iv) any combination of (i) to (iii).

For instance, in some aspects, the GALR2 agonist comprises, consists of, or consists essentially of the amino acid sequence X¹WX³X⁴X⁵X⁶X⁷X⁸YLX¹¹X¹²X¹³X¹⁴ (SEQ ID NO: 1), wherein: (i) X¹ is a D-amino acid, (ii) the W at position 2 of SEQ ID NO: 1 is D-tryptophan, (iii) X⁴ is a D-amino acid, (iv) X⁶ is a D-amino acid, (v) X¹¹ is a D-amino acid, (vi) X¹² is a D-amino acid, (vii) X¹³ is a D-amino acid, (viii) X¹⁴ is a D-amino acid, or (ix) any combination of (i) to (viii). More specifically, in some aspects, the GALR2 agonist comprises, consists of, or consists essentially of the amino acid sequence X¹WX³X⁴X⁵X⁶X⁷X⁸YLX¹¹X¹²X¹³X¹⁴ (SEQ ID NO: 1), wherein: (i) X¹ is D-asparagine, (ii) the W at position 2 of SEQ ID NO: 1 is D-tryptophan, (iii) X⁴ is D-alanine or D-valine, (iv) X⁶ is D-alanine, (v) X¹¹ is D-lysine, (vi) X¹² is D-alanine, (vii) X¹³ is D-alanine, (viii) X¹⁴ is D-glutamine, or (ix) any combination of (i) to (viii). In some aspects, the GALR2 agonist comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X¹ is D-asparagine, (2) X⁴ is D-alanine, (3) X¹⁴ is D-glutamine, or (4) any combination thereof. Non-limiting example of such a GALR2 agonist is provided in SEQ ID NO: 87. In some aspects, the GALR2 agonist comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X⁴ is D-glutamate, (2) X¹⁴ is D-glutamine, or (3) X⁴ is D-glutamate and X¹⁴ is D-glutamine. Non-limiting example of such a GALR2 agonist is provided in SEQ ID NO: 90. In some aspects, the GALR2 agonist comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X⁴ is D-arginine, (2) X¹⁴ is D-glutamine, or (3) X⁴ is D-arginine and X¹⁴ is D-glutamine. Non-limiting example of such a GALR2 agonist is provided in SEQ ID NO: 91.

In some aspects, a GALR2 agonist useful for the present disclosure comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E), arginine (R), alanine (A), or valine (V); (4) X⁵ is asparagine (N) or glutamine (Q); (5) X⁶ is alanine (A) or serine(S); (6) X⁷ is alanine (A); (7) X⁸ is leucine (L), glutamine (Q), or glycine (G); (8) X¹¹ is phenylalanine (F); (9) X¹² is glycine (G) or alanine (A); (10) X¹³ is proline (P), arginine (R), or alanine (A); (11) X¹⁴ is glutamine (Q), histidine (H), or valine (V); or (12) any combination of (1) to (11). For instance, in some aspects, a GALR2 agonist useful for the present disclosure comprises, consists of, or consists essentially of the amino acid sequence NWTPQAALYLFGAQ (SEQ ID NO: 48).

In some aspects, a GALR2 agonist useful for the present disclosure comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E), arginine (R), alanine (A), or valine (V); (4) X⁵ is asparagine (N); (5) X⁶ is alanine (A) or serine(S); (6) X⁷ is alanine (A); (7) X⁸ is leucine (L), glutamine (Q), or glycine (G); (8) X¹¹ is phenylalanine (F); (9) X¹² is glycine (G) or alanine (A); (10) X¹³ is proline (P), arginine (R), or alanine (A); (11) X¹⁴ is glutamine (Q), histidine (H), or valine (V); or (12) any combination of (1) to (11). In some aspects, such a GALR2 agonist comprises, consists of, or consists essentially of the amino acid sequence NWTPNAALYLFGAQ (SEQ ID NO: 50).

In some aspects, a GALR2 agonist useful for the present disclosure comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E), arginine (R), alanine (A), or valine (V); (4) X⁵ is asparagine (N); (5) X⁶ is alanine (A) or serine(S); (6) X⁷ is alanine (A); (7) X⁸ is leucine (L), glutamine (Q), or glycine (G); (8) X¹¹ is phenylalanine (F); (9) X¹² is glycine (G) or alanine (A); (10) X¹³ is proline (P); (11) X¹⁴ is glutamine (Q), histidine (H), or valine (V); or (12) any combination of (1) to (11). In some aspects, such a GALR2 agonist comprises, consists of, or consists essentially of the amino acid sequence NWTPNAALYLFGPQ (SEQ ID NO: 51).

In some aspects, a GALR2 agonist that can be used with the present disclosure comprises, consists of, or consists essentially of the amino acid sequence set forth in any of the following: SEQ ID NO: 9, SEQ ID NO: 10, SEQ ID NO: 11, SEQ ID NO: 12, SEQ ID NO: 13, SEQ ID NO: 14, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 31, SEQ ID NO: 32, SEQ ID NO: 33, SEQ ID NO: 37, SEQ ID NO: 39, SEQ ID NO: 40, SEQ ID NO: 41, SEQ ID NO: 42, SEQ ID NO: 44, SEQ ID NO: 45, SEQ ID NO: 47, SEQ ID NO: 48, SEQ ID NO: 50, SEQ ID NO: 51; SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 87, SEQ ID NO: 88, SEQ ID NO: 89, SEQ ID NO: 90, or SEQ ID NO: 91. See Table 4 (below)

TABLE 4
Exemplary GALR2 Agonists

| SEQ ID | | |
|---|---|---|
| NO: | Modification | Sequence |
| 9 | [dN1]-GALR2 | [dN¹]W²T³P⁴Q⁵A⁶M⁷L⁸Y⁹L¹⁰K¹¹G¹²A¹³Q¹⁴ |
| | Agonist | |
| 10 | [pQ1]-GALR2 | [pQ¹]W²T³P⁴Q⁵A⁶M⁷L⁸Y⁹L¹⁰K¹¹G¹²A¹³Q¹⁴ |
| | Agonist | |
| 11 | [Ac-N1]- | [Ac-N¹]W²T³P⁴Q⁵A⁶M⁷L⁸Y⁹L¹⁰K¹¹G¹²A¹³Q¹⁴ |
| | GALR2 Agonist | |
| 12 | [G1]-GALR2 | [G¹]W²T³P⁴Q⁵A⁶M⁷L⁸Y⁹L¹⁰K¹¹G¹²A¹³Q¹⁴ |
| | Agonist | |
| 13 | [dW2]-GALR2 | N¹[dW²]T³P⁴Q⁵A⁶M⁷L⁸Y⁹L¹⁰K¹¹G¹²A¹³Q¹⁴ |
| | Agonist | |
| 14 | [2NaI2]-GALR2 | N¹[2NaI²]T³P⁴Q⁵A⁶M⁷L⁸Y⁹L¹⁰K¹¹G¹²A¹³Q¹⁴ |
| | Agonist | |
| 16 | [A3]-GALR2 | N¹W²[A³]P⁴Q⁵A⁶M⁷L⁸Y⁹L¹⁰K¹¹G¹²A¹³Q¹⁴ |
| | Agonist | |
| 17 | [K3]-GALR2 | N¹W²[K³]P⁴Q⁵A⁶M⁷L⁸Y⁹L¹⁰K¹¹G¹²A¹³Q¹⁴ |
| | Agonist | |
| 18 | [dA4]-GALR2 | N¹W²T³[dA⁴]Q⁵A⁶M⁷L⁸Y⁹L¹⁰K¹¹G¹²A¹³Q¹⁴ |
| | Agonist | |
| 19 | [E4]-GALR2 | N¹W²T³[E⁴]Q⁵A⁶M⁷L⁸Y⁹L¹⁰K¹¹G¹²A¹³Q¹⁴ |
| | Agonist | |
| 20 | [L4]-GALR2 | N¹W²T³[L⁴]Q⁵A⁶M⁷L⁸Y⁹L¹⁰K¹¹G¹²A¹³Q¹⁴ |
| | Agonist | |
| 21 | [R4]-GALR2 | N¹W²T³[R⁴]Q⁵A⁶M⁷L⁸Y⁹L¹⁰K¹¹G¹²A¹³Q¹⁴ |
| | Agonist | |
| 22 | [dV4]-GALR2 | N¹W²T³[dV⁴]Q⁵A⁶M⁷L⁸Y⁹L¹⁰K¹¹G¹²A¹³Q¹⁴ |
| | Agonist | |
| 23 | [N5]-GALR2 | N¹W²T³P⁴[N⁵]A⁶M⁷L⁸Y⁹L¹⁰K¹¹G¹²A¹³Q¹⁴ |
| | Agonist | |
| 24 | [dA6]-GALR2 | N¹W²T³P⁴Q⁵[dA⁶]M⁷L⁸Y⁹L¹⁰K¹¹G¹²A¹³Q¹⁴ |
| | Agonist | |
| 25 | [A7]-GALR2 | N¹W²T³P⁴Q⁵A⁶[A⁷]L⁸Y⁹L¹⁰K¹¹G¹²A¹³Q¹⁴ |
| | Agonist | |
| 26 | [G8]-GALR2 | N¹W²T³P⁴Q⁵A⁶M⁷[G⁸]Y⁹L¹⁰K¹¹G¹²A¹³Q¹⁴ |
| | Agonist | |
| 27 | [Q8]-GALR2 | N¹W²T³P⁴Q⁵A⁶M⁷[Q⁸]Y⁹L¹⁰K¹¹G¹²A¹³Q¹⁴ |
| | Agonist | |
| 31 | [F11]-GALR2 | N¹W²T³P⁴Q⁵A⁶M⁷L⁸Y⁹L¹⁰[F¹¹]G¹²A¹³Q¹⁴ |
| | Agonist | |
| 32 | [L11]-GALR2 | N¹W²T³P⁴Q⁵A⁶M⁷L⁸Y⁹L¹⁰[L¹¹]G¹²A¹³Q¹⁴ |
| | Agonist | |
| 33 | [Y11]-GALR2 | N¹W²T³P⁴Q⁵A⁶M⁷L⁸Y⁹L¹⁰[Y¹¹]G¹²A¹³Q¹⁴ |
| | Agonist | |
| 37 | [dA12]-GALR2 | N¹W²T³P⁴Q⁵A⁶M⁷L⁸Y⁹L¹⁰K¹¹[dA¹²]A¹³Q¹⁴ |
| | Agonist | |
| 39 | [P13]-GALR2 | N¹W²T³P⁴Q⁵A⁶M⁷L⁸Y⁹L¹⁰K¹¹G¹²[P¹³]Q¹⁴ |
| | Agonist | |
| 40 | [dQ14]-GALR2 | N¹W²T³P⁴Q⁵A⁶M⁷L⁸Y⁹L¹⁰K¹¹G¹²A¹³[dQ¹⁴] |
| | Agonist | |
| 41 | [H14]-GALR2 | N¹W²T³P⁴Q⁵A⁶M⁷L⁸Y⁹L¹⁰K¹¹G¹²A¹³[H¹⁴] |
| | Agonist | |
| 42 | [PEG]-GALR2 | PEG-N¹W²T³P⁴Q⁵A⁶M⁷L⁸Y⁹L¹⁰K¹¹G¹²A¹³Q¹⁴ |
| | Agonist | |
| 44 | [Cit1]-GALR2 | [Cit¹]W²T³P⁴Q⁵A⁶M⁷L⁸Y⁹L¹⁰K¹¹G¹²A¹³Q¹⁴ |
| | Agonist | |
| 45 | [Fmoc]-GALR2 | [Fmoc¹]W²T³P⁴Q⁵A⁶M⁷L⁸Y⁹L¹⁰K¹¹G¹²A¹³Q¹⁴ |
| | Agonist | |
| 47 | [SPX-M40]- | N¹W²T³P⁴Q⁵A⁶M⁷L⁸Y⁹L¹⁰K¹¹G¹²A¹³[P¹⁴P¹⁵A¹⁶L¹⁷A¹⁸L¹⁹A²⁰] |
| | GALR2 Agonist | |
| 48 | [A7F11]- | N¹W²T³P⁴Q⁵A⁶[A⁷]L⁸Y⁹L¹⁰[F¹¹]G¹²A¹³Q¹⁴ |
| | GALR2 Agonist | |
| 50 | [N5A7F11]- | N¹W²T³P⁴[N⁵]A⁶[A⁷]L⁸Y⁹L¹⁰[F¹¹]G¹²A¹³Q¹⁴ |
| | GALR2 Agonist | |
| 51 | [N5A7F11P13]- | N¹W²T³P⁴[N⁵]A⁶[A⁷]L⁸Y⁹L¹⁰[F¹¹]G¹²[P¹³]Q¹⁴ |
| | GALR2 Agonist | |
| 52 | [N5A7F11H14]- | N¹W²T³P⁴[N⁵]A⁶[A⁷]L⁸Y⁹L¹⁰[F¹¹]G¹²A¹³[H¹⁴] |
| | GALR2 Agonist | |
| 53 | [PEG2]-GALR2 | PEG-N¹W²T³P⁴[N⁵]A⁶[A⁷]L⁸Y⁹L¹⁰[F¹¹]G¹²A¹³[H¹⁴] |
| | Agonist | |
| 54 | [3-NO2-Y9]- | N¹W²T³P⁴Q⁵A⁶M⁷L⁸[X⁹]L¹⁰K¹¹G¹²A¹³Q¹⁴; (X = Y(NO2I)) |
| | GALR2 Agonist | |
| 55 | [Fmoc-Qu]- | [Fmoc¹]W²T³P⁴[N⁵]A⁶[A⁷]L⁸Y⁹L¹⁰[F¹¹]G¹²[P¹³]Q¹⁴ |
| | GALR2 Agonist | |
| 56 | [Fmoc-Qu- | Fmoc-N¹W²T³P⁴[N⁵]A⁶[A⁷]L⁸Y⁹L¹⁰[F¹¹]G¹²[P¹³][dQ¹⁴] |
| | dQ14]-GALR2 | |
| | Agonist | |
| 57 | [Fmoc-Qu- | Fmoc-N¹W²T³[dA⁴][N⁵]A⁶[A⁷]L⁸Y⁹L¹⁰[F¹¹]G¹²[P¹³]Q¹⁴ |
| | dA4]-GALR2 | |
| | Agonist | |
| 58 | [Fmoc-Qu-dA4- | Fmoc-N¹W²T³[dA⁴][N⁵]A⁶[A⁷]L⁸Y⁹L¹⁰[F¹¹]G¹²[P¹³][dQ¹⁴] |
| | dQ14]-GALR2 | |
| | Agonist | |
| 59 | [PEG-Qu-dA4- | PEG-N¹W²T³[dA⁴][N⁵]A⁶[A⁷]L⁸Y⁹L¹⁰[F¹¹]G¹²[P¹³][dQ¹⁴] |
| | dQ14]-GALR2 | |
| | Agonist | |
| 87 | [dN1-dA4- | [dN¹]W²T³[dA]⁴[N⁵][A⁶][A⁷]L⁸Y⁹L¹⁰[F¹¹]G¹²[P¹³][dQ¹⁴] |
| | dQ14]-GALR2 | |
| | Agonist | |
| 88 | [PEG-dE4- | PEG-N¹W²T³[dE]⁴[N⁵][A⁶][A⁷]L⁸Y⁹L¹⁰[F¹¹]G¹²[P¹³][dQ¹⁴] |
| | dQ14]-GALR2 | |
| | Agonist | |
| 89 | [PEG-dR4- | PEG-N¹W²T³[dR]⁴[N⁵][A⁶][A⁷]L⁸Y⁹L¹⁰[F¹¹]G¹²[P¹³][dQ¹⁴] |
| | dQ14]-GALR2 | |
| | Agonist | |
| 90 | [dE4-dQ14]- | N¹W²T³[dE]⁴[N⁵][A⁶][A⁷]L⁸Y⁹L¹⁰[F¹¹]G¹²[P¹³][dQ¹⁴] |
| | GALR2 Agonist | |
| 91 | [dR4-dQ14]- | N¹W²T³[dR]⁴[N⁵][A⁶][A⁷]L⁸Y⁹L¹⁰[F¹¹]G¹²[P¹³][dQ¹⁴] |
| | GALR2 Agonist | |

In some aspects, the GALR2 agonist comprises, consists of, or consists essentially of the amino acid sequence set forth in SEQ ID NO: 9. In some aspects, the GALR2 agonist comprises, consists of, or consists essentially of the amino acid sequence set forth in SEQ ID NO: 10. In some aspects, the GALR2 agonist comprises, consists of, or consists essentially of the amino acid sequence set forth in SEQ ID NO: 11. In some aspects, the GALR2 agonist comprises, consists of, or consists essentially of the amino acid sequence set forth in SEQ ID NO: 12. In some aspects, the GALR2 agonist comprises, consists of, or consists essentially of the amino acid sequence set forth in SEQ ID NO: 13. In some aspects, the GALR2 agonist comprises, consists of, or consists essentially of the amino acid sequence set forth in SEQ ID NO: 14. In some aspects, the GALR2 agonist comprises, consists of, or consists essentially of the amino acid sequence set forth in SEQ ID NO: 16. In some aspects, the GALR2 agonist comprises, consists of, or consists essentially of the amino acid sequence set forth in SEQ ID NO: 17. In some aspects, the GALR2 agonist comprises, consists of, or consists essentially of the amino acid sequence set forth in SEQ ID NO: 18. In some aspects, the GALR2 agonist comprises, consists of, or consists essentially of the amino acid sequence set forth in SEQ ID NO: 19. In some aspects, the GALR2 agonist comprises, consists of, or consists essentially of the amino acid sequence set forth in SEQ ID NO: 20. In some aspects, the GALR2 agonist comprises, consists of, or consists essentially of the amino acid sequence set forth in SEQ ID NO: 21. In some aspects, the GALR2 agonist comprises, consists of, or consists essentially of the amino acid sequence set forth in SEQ ID NO: 22. In some aspects, the GALR2 agonist comprises, consists of, or consists essentially of the amino acid sequence set forth in SEQ ID NO: 23. In some aspects, the GALR2 agonist comprises, consists of, or consists essentially of the amino acid sequence set forth in SEQ ID NO: 24. In some aspects, the GALR2 agonist comprises, consists of, or consists essentially of the amino acid sequence set forth in SEQ ID NO: 25. In some aspects, the GALR2 agonist comprises, consists of, or consists essentially of the amino acid sequence set forth in SEQ ID NO: 26. In some aspects, the GALR2 agonist comprises, consists of, or consists essentially of the amino acid sequence set forth in SEQ ID NO: 27. In some aspects, the GALR2 agonist comprises, consists of, or consists essentially of the amino acid sequence set forth in SEQ ID NO: 31. In some aspects, the GALR2 agonist comprises, consists of, or consists essentially of the amino acid sequence set forth in SEQ ID NO: 32. In some aspects, the GALR2 agonist comprises, consists of, or consists essentially of the amino acid sequence set forth in SEQ ID NO: 33. In some aspects, the GALR2 agonist comprises, consists of, or consists essentially of the amino acid sequence set forth in SEQ ID NO: 37. In some aspects, the GALR2 agonist comprises, consists of, or consists essentially of the amino acid sequence set forth in SEQ ID NO: 39. In some aspects, the GALR2 agonist comprises, consists of, or consists essentially of the amino acid sequence set forth in SEQ ID NO: 40. In some aspects, the GALR2 agonist comprises, consists of, or consists essentially of the amino acid sequence set forth in SEQ ID NO: 41. In some aspects, the GALR2 agonist comprises, consists of, or consists essentially of the amino acid sequence set forth in SEQ ID NO: 42. In some aspects, the GALR2 agonist comprises, consists of, or consists essentially of the amino acid sequence set forth in SEQ ID NO: 44. In some aspects, the GALR2 agonist comprises, consists of, or consists essentially of the amino acid sequence set forth in SEQ ID NO: 45. In some aspects, the GALR2 agonist comprises, consists of, or consists essentially of the amino acid sequence set forth in SEQ ID NO: 47. In some aspects, the GALR2 agonist comprises, consists of, or consists essentially of the amino acid sequence set forth in SEQ ID NO: 48. In some aspects, the GALR2 agonist comprises, consists of, or consists essentially of the amino acid sequence set forth in SEQ ID NO: 50. In some aspects, the GALR2 agonist comprises, consists of, or consists essentially of the amino acid sequence set forth in SEQ ID NO: 51. In some aspects, the GALR2 agonist comprises, consists of, or consists essentially of the amino acid sequence set forth in SEQ ID NO: 52. In some aspects, the GALR2 agonist comprises, consists of, or consists essentially of the amino acid sequence set forth in SEQ ID NO: 53. In some aspects, the GALR2 agonist comprises, consists of, or consists essentially of the amino acid sequence set forth in SEQ ID NO: 54. In some aspects, the GALR2 agonist comprises, consists of, or consists essentially of the amino acid sequence set forth in SEQ ID NO: 55. In some aspects, the GALR2 agonist comprises, consists of, or consists essentially of the amino acid sequence set forth in SEQ ID NO: 56. In some aspects, the GALR2 agonist comprises, consists of, or consists essentially of the amino acid sequence set forth in SEQ ID NO: 57. In some aspects, the GALR2 agonist comprises, consists of, or consists essentially of the amino acid sequence set forth in SEQ ID NO: 58. In some aspects, the GALR2 agonist comprises, consists of, or consists essentially of the amino acid sequence set forth in SEQ ID NO: 59. In some aspects, the GALR2 agonist comprises, consists of, or consists essentially of the amino acid sequence set forth in SEQ ID NO: 87. In some aspects, the GALR2 agonist comprises, consists of, or consists essentially of the amino acid sequence set forth in SEQ ID NO: 88. In some aspects, the GALR2 agonist comprises, consists of, or consists essentially of the amino acid sequence set forth in SEQ ID NO: 89. In some aspects, the GALR2 agonist comprises, consists of, or consists essentially of the amino acid sequence set forth in SEQ ID NO: 90. In some aspects, the GALR2 agonist comprises, consists of, or consists essentially of the amino acid sequence set forth in SEQ ID NO: 91.

As described and demonstrated herein, in some aspects, one or more of the amino acid modifications described above allows a GALR2 agonist described herein to exhibit one or more properties that are not present in other GALR2 ligands (e.g., wild-type spexin and/or galanin).

For instance, in some aspects, a GALR2 agonist described herein is capable of inducing the activation of GALR2 and GALR3 but not GALR1. Compared to a reference (e.g., wild-type spexin and/or galanin), in some aspects, a GALR2 agonist described herein exhibits greater potency (or agonistic effect) (e.g., EC₅₀) towards GALR2. For instance, in some aspects, compared to the reference (e.g., wild-type spexin and/or galanin), the potency of a GALR2 agonist described herein towards GALR2 is greater than about 1-fold, greater than about 2-fold, greater than about 3-fold, greater than about 4-fold, greater than about 5-fold, greater than about 6-fold, greater than about 7-fold, greater than about 8-fold, greater than about 9-fold, greater than about 10-fold, greater than about 15-fold, greater than about 20-fold, greater than about 25-fold, greater than about 30-fold, greater than about 35-fold, greater than about 40-fold, greater than about 45-fold, greater than about 50-fold, greater than about 75-fold, or greater than about 100-fold. In some aspects, a GALR2 agonist described herein is capable of inducing GALR2 activation with a potency (EC₅₀) of less than about −5 nM, less than about −6 nM, less than about −7 nM, less than about −8 nM, or less than about −9 nM.

In some aspects, unlike other GALR2 ligands (e.g., wild-type spexin and/or galanin), a GALR2 agonist described herein is specific to GALR2. As used herein, an agonist is “specific to GALR2” where the agonist is capable of primarily inducing the activation of GALR2 as compared to the other GALR subtypes (GALR1 and GALR3). As is apparent from the present disclosure, in some aspects, an agonist that is specific to GALR2 can have some agonistic effect on GALR3. However, compared to the agonistic effect on GALR2, such agonists exhibit much reduced agonistic effect on GALR3. Accordingly, in some aspects, compared to the agonistic effect on GALR3, the agonistic effect of a GALR2 agonist described herein on GALR2 is greater than about 1-fold, greater than about 2-fold, greater than about 3-fold, greater than about 4-fold, greater than about 5-fold, greater than about 6-fold, greater than about 7-fold, greater than about 8-fold, greater than about 9-fold, greater than about 10-fold, greater than about 15-fold, greater than about 20-fold, greater than about 25-fold, greater than about 30-fold, greater than about 35-fold, greater than about 40-fold, greater than about 45-fold, greater than about 50-fold, greater than about 75-fold, or greater than about 100-fold. In some aspects, an agonist that is specific to GALR2 (e.g., an GALR2 agonist described herein) does not induce the activation of GALR3. Accordingly, in some aspects, GALR2 agonists described herein does not induce the activation of both GALR1 and GALR3. Unless indicated otherwise, a GALR2 agonist does not induce the activation of a GALR subtype (GALR1 and/or GALR3) where the potency (EC₅₀) of the GALR2 agonist to the GALR subtype is greater than about −5 nM.

### III.B. Other Modifications

As is apparent from at least the above disclosures, in some aspects, GALR2 agonists useful for the present disclosure comprises one or more modifications at the N-terminal end, at the C-terminal end, or both at the N-terminal end and at the C-terminal end. In some aspects, such modifications can help increase the stability of the GALR2 agonists. In some aspects, such modifications at the N-terminal end and/or the C-terminal end do not affect the activity of the GALR2 agonist. Instead, such modifications at the N-terminal end and/or the C-terminal end aid in the synthesis/production of the GALR2 agonists. For instance, in some aspects, the N-terminal end and/or the C-terminal end can improve the solubility of the GALR2 agonists. In some aspects, the modifications at the N-terminal end and/or the C-terminal end prevent degradation in the blood, e.g., when administered to a subject. Accordingly, in some aspects, the modifications at the N-terminal end and/or the C-terminal end could be useful in increasing the half-life of the GALR2 agonist. In some aspects, a GALR2 agonist described herein has been modified (or engineered) such that the GALR2 agonist is conjugated to a N-terminal protecting group. Any suitable N-terminal protecting group known in the art can be used. In some aspects, a GALR2 agonist described herein is conjugated at the N-terminal end by any of the following: 9-fluorenylmethoxycarbonyl group (Fmoc group), pyroglutamate (pQ), citrulline (Cit), acetyl (Ac) group, polyethylene glycol (PEG), or a combination thereof. In some aspects, the N-terminal end of a GALR2 agonist described herein can be methylated.

Accordingly, in some aspects, a GALR2 agonist described herein comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E), arginine (R), alanine (A), or valine (V); (4) X⁵ is asparagine (N) or glutamine (Q); (5) X⁶ is alanine (A) or serine(S); (6) X⁷ is alanine (A) or methionine (M); (7) X⁸ is leucine (L), glutamine (Q), or glycine (G); (8) X¹¹ is leucine (L), phenylalanine (F), tyrosine (Y), or aspartic acid (D); (9) X¹² is glycine (G) or alanine (A); (10) X¹³ is proline (P), arginine (R), or alanine (A); (11) X¹⁴ is glutamine (Q), histidine (H), or valine (V); or (12) any combination of (1) to (11), and wherein the N-terminal end of the GALR2 agonist is conjugated to any of the following: Fmoc group, pQ, Cit, Ac group, PEG, or a combination thereof. In some aspects, the N-terminal end of the GALR2 agonist is conjugated to Fmoc. In some aspects, the N-terminal end of the GALR2 agonist is conjugated to pQ. In some aspects, the N-terminal end of the GALR2 agonist is conjugated to an Ac group. In some aspects, the N-terminal end of the GALR2 agonist is conjugated to PEG. Non-limiting examples of such GALR2 agonists are illustrated in Table 4 (above)—see, e.g., SEQ ID NOs: 10, 11, 42, 44, 45, 53, 55-59, and 87-89.

In some aspects, the C-terminal end of a GALR2 agonist has been modified (or engineered) such that the GALR2 agonist is conjugated to a C-terminal protecting group. Any suitable C-terminal protecting group known in the art can be used. Non-limiting examples of such C-terminal protecting group include: an amine group (—NH₂), strep-tags, His-tags, or combinations thereof. In some aspects, the C-terminal end of a GALR2 agonist is conjugated to a C-terminal protecting group during synthesis but is then subsequently removed after synthesis.

In some aspects, a GALR2 agonist described herein has been further modified, such that the GALR2 agonist exhibits increased survival or half-life, e.g., when administered to a subject. Accordingly, in some aspects, a GALR2 agonist described herein is conjugated to a half-life extending moiety. For instance, in some aspects, a GALR2 agonist described herein comprises, consists of, or consists essentially of the amino acid sequence X¹WX³X⁴X⁵X⁶X⁷X⁸YLX¹¹X¹²X¹³X¹⁴ (SEQ ID NO: 1), wherein: (1) X¹ is asparagine (N), glycine (G), pyroglutamate (pQ), or citrulline (Cit); (2) X³ is threonine (T), alanine (A), or lysine (K); (3) X⁴ is proline (P), leucine (L), glutamate (E), arginine (R), alanine (A), or valine (V); (4) X⁵ is asparagine (N) or glutamine (Q); (5) X⁶ is alanine (A) or serine(S); (6) X⁷ is alanine (A) or methionine (M); (7) X⁸ is leucine (L), glutamine (Q), or glycine (G); (8) X¹¹ is leucine (L), phenylalanine (F), tyrosine (Y), or aspartic acid (D); (9) X¹² is glycine (G) or alanine (A); (10) X¹³ is proline (P), arginine (R), or alanine (A); (11) X¹⁴ is glutamine (Q), histidine (H), or valine (V); or (12) any combination of (1) to (11), and wherein the GALR2 agonist is conjugated to a half-life extending moiety.

Any suitable half-life extending moieties known in the art can be used with the present disclosure. Non-limiting examples of such half-life extending moieties include: a Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof. In some aspects, the half-life extending moiety is Fc.

In some aspects, a GALR2 agonist useful for the present disclosure can comprise one or more additional moieties that allow the molecule to be specifically targeted to different tissues, e.g., when administered to a subject. For instance, in some aspects, a GALR2 agonist described herein can comprise a peptide that allows the agonist to penetrate across the blood-brain barrier (also referred to herein as “BBB shuttles”). Examples of such BBB shuttles are known in the art. Non-limiting examples are provided in Table 5 (below). See, e.g., Oller-Salvia et al., Chem Soc Rev 45:4690 (2016).

TABLE 5
BBB Shuttles

| SEQ | | |
|---|---|---|
| ID NO | Peptide | Sequence |
| 60 | Angiopep-2 | TFFYGGSRGKRNNFKTEEY-OH |
| 61 | ApoB (3371-3409) | SSVIDALQYKLEGTTRLTRKRGLKLATALSLSNKFVEGS |
| 62 | ApoE (159-167)₂ | (LRKLRKRLL)₂ |
| 63 | Peptide-22 | Ac-C(&)MPRLRGC(&)-NH₂ |
| 64 | THR | THRPPMWSPVWP-NH₂ |
| 65 | THR retro-enantio | pwvpswmpprht-NH₂ |
| 66 | CRT | C(&)RTIGPSVC(&) |
| 67 | Leptin30 | YQQILTSMPSRNVIQISNDLENLRDLLHVL |
| 68 | RVG29 | YTIWMPENPRPGTPCDIFTNSRGKRASNG-OH |
| 69 | CDX | GreirtGraerwsekf-OH |
| 70 | Apamin | C(&1)NC(&₂)KAPETALC(&₁)-ARRC(&₂)QQH-NH₂ |
| 71 | MiniAp-4 | [Dap](&)KAPETALD(&) |
| 72 | GSH | γ-_{L}-glutamyl-CG-OH |
| 73 | G23 | HLNILSTLWKYRC |
| 74 | g7 | GFtGFLS(O-b-Glc)-NH₂ |
| 75 | TGN | TGNYKALHPHNG |
| 76 | TAT (47-57) | YGRKKRRQRRR-NH₂ |
| 77 | SynB1 | RGGRLSYSRRRFSTSTGR |
| 78 | Diketopiperazines | &(N-MePhe)-(N-MePhe)Diketopiperazines |
| 79 | PhPro | (Phenylproline)4-NH₂ |

Nomenclature for cyclic peptides (&) is adapted to the 3-letter amino acid code from the one described in Spengler et al., J Pept Res 65: 550-555 (2005); [Dap] stands for diaminopropionic acid.

### IV. Nucleic Acids, Vectors, and Cells

Some aspects of the present disclosure are related to one or more nucleic acid molecules (also referred to herein as “nucleic acids” or derivatives thereof) that encode a GALR2 agonist described herein. The nucleic acids can be present in whole cells, in a cell lysate, or in a partially purified or substantially pure form. In some aspects, the nucleic acid is a DNA sequence and/or an RNA sequence (e.g., mRNA). In some aspects, the nucleic acids comprise a modified nucleotide analog. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. In some aspects, a nucleic acid molecule can or cannot contain intronic sequences. In some aspects, the nucleic acid is a cDNA molecule. Nucleic acids described herein can be obtained using standard molecular biology techniques known in the art.

In some aspects, the present disclosure relates to a vector comprising an isolated nucleic acid molecule encoding a GALR2 agonist described herein. Suitable vectors for the disclosure include, but are not limited to, expression vectors, viral vectors, and plasmid vectors. In some aspects, the vector is a viral vector.

As used herein, an “expression vector” refers to any nucleic acid construct which contains the necessary elements for the transcription and translation of an inserted coding sequence, or in the case of a RNA viral vector, the necessary elements for replication and translation, when introduced into an appropriate host cell. Expression vectors can include plasmids, phagemids, viruses, and derivatives thereof.

As used herein, “viral vectors” include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, WO9712622, WO9817815, WO9817816, and WO9818934, each which is incorporated herein by reference in its entirety.

Other vectors include plasmid vectors. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Additionally, plasmids can be custom designed using standard molecular biology techniques to remove and/or add specific fragments of DNA.

Also encompassed herein are cells comprising a nucleic acid molecule encoding a GALR2 agonist described herein. In some aspects, the cells comprise a vector comprising the nucleic acid molecule. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### V. Pharmaceutical Compositions

Some aspects of the present disclosure are directed to compositions comprising a therapeutic agent described herein (e.g., GALR2 agonist, nucleic acid encoding a GALR2 agonist, vector comprising the nucleic acid, and/or cell comprising the vector) having the desired degree of purity in a physiologically acceptable carrier, excipient or stabilizer (Remington's Pharmaceutical Sciences (1990) Mack Publishing Co., Easton, PA). For instance, in some aspects, provided herein is a composition comprising (i) a GALR2 agonist and (ii) a pharmaceutically acceptable carrier, excipient, or stabilizer. In some aspects, provided herein is a composition comprising a nucleic acid molecule encoding a GALR2 agonist and (ii) a pharmaceutically acceptable carrier, excipient, or stabilizer. In some aspects, provided herein is a composition comprising a vector, which comprises a nucleic acid molecule encoding a GALR2 agonist and (ii) a pharmaceutically acceptable carrier, excipient, or stabilizer. In some aspects, provided herein is a composition comprising a cell, which has been modified to comprise a vector comprising a nucleic acid molecule encoding a GALR2 agonist and (ii) a pharmaceutically acceptable carrier, excipient, or stabilizer.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN® PLURONICS® or polyethylene glycol (PEG).

In some aspects, a pharmaceutical composition useful for the present disclosure comprises any of the therapeutic agents described herein (e.g., a GALR2 agonist described herein, a nucleic acid molecule encoding the GALR2 agonist, a vector comprising the nucleic acid, and/or cell modified to comprise the vector), and optionally one or more additional prophylactic or therapeutic agents, in a pharmaceutically acceptable carrier. In some aspects, pharmaceutical compositions comprise any of the therapeutic agents described herein (e.g., a GALR2 agonist described herein, a nucleic acid molecule encoding the GALR2 agonist, a vector comprising the nucleic acid, and/or cell modified to comprise the vector), and optionally one or more additional prophylactic of therapeutic agents, in a pharmaceutically acceptable carrier. In some aspects, the therapeutic agents described herein are the only active ingredient included in the pharmaceutical composition.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

A pharmaceutical composition can be formulated for any route of administration to a subject. Specific examples of routes of administration include intranasal, oral, parenterally, intrathecally, intra-cerebroventricularly, pulmonarily, subcutaneously, or intraventricularly. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Preparations for parenteral administration of a therapeutic agent described herein (e.g., GALR2 agonist, nucleic acid encoding a GALR2 agonist, vector comprising the nucleic acid, and/or cell comprising the vector) include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising a therapeutic agent are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

A pharmaceutical composition can be formulated as an aerosol for topical application, such as by inhalation (see, e.g., U.S. Pat. Nos. 4,044,126, 4,414,209 and 4,364,923). These formulations for administration to the respiratory tract can be in the form of an aerosol or solution for a nebulizer, or as a microfine powder for insufflations, alone or in combination with an inert carrier such as lactose. In such a case, the particles of the formulation can, in some aspects, have diameters of less than about 50 microns, e.g., less than about 10 microns.

A pharmaceutical composition can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer any of the therapeutic agents described herein. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

In some aspects, a pharmaceutical composition described herein is a lyophilized powder, which can be reconstituted for administration as solutions, emulsions and other mixtures. It can also be reconstituted and formulated as solids or gels. The lyophilized powder is prepared by dissolving any of the therapeutic agents described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium, or potassium phosphate or other such buffer known to those of skill in the art. In some aspects, the buffer is at about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of any of the therapeutic agents described herein (e.g., GALR2 agonist, nucleic acid encoding a GALR2 agonist, vector comprising the nucleic acid, and/or cell comprising the vector). The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

In some aspects, a pharmaceutical composition comprising any of the therapeutic agents described herein can also be formulated to be targeted to a particular tissue, receptor, or other area of the body of the subject to be treated. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542, and 5,709,874.

The compositions to be used for in vivo administration can be sterile. In some aspects, this can be accomplished by filtration through, e.g., sterile filtration membranes.

### VI. Kits

Also provided herein are kits comprising one or more of the therapeutic agents described herein (e.g., GALR2 agonist, nucleic acid encoding a GALR2 agonist, vector comprising the nucleic acid, and/or cell comprising the vector). As is apparent from the present disclosure, in some aspects, a kit comprising a GALR2 agonist described herein can be useful in treating various diseases or disorders (e.g., gastrointestinal disorder and/or endocrine disorder). In some aspects, provided herein is a pharmaceutical pack or kit comprising one or more containers filled with one or more of the ingredients of the pharmaceutical compositions described herein, optional an instruction for use. In some aspects, the kits contain a pharmaceutical composition described herein and any prophylactic or therapeutic agent, such as those described herein.

The following examples are offered by way of illustration and not by way of limitation.

### EXAMPLES

### Example 1: Colonic Transit Time in Naïve Mice Treated with GALR2 Agonist

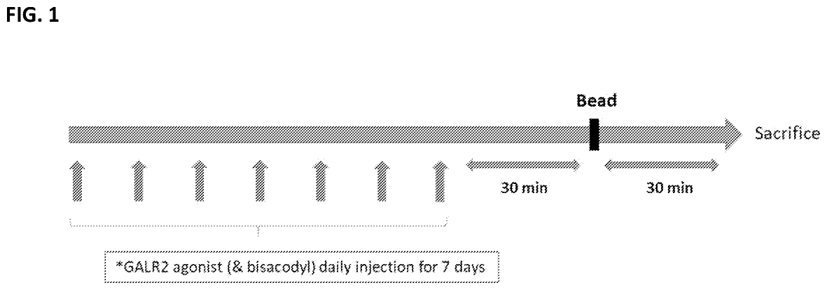

To begin assessing the therapeutic effects of GALR2 agonists described herein, a pegylated GALR2 (PEG-GALR2) agonist was administered daily for seven days to naïve mice (i.e., no OIC) as described in FIG. 1. PEG-GALR2 was administered to the animals either intraperitoneally (1 mg/kg) or intranasally (10 μg/head). Control animals received either vehicle control (PBS) (negative control) or bisacodyl (positive control). The different treatment groups are provided in Table 6 (below).

TABLE 6
Experimental Groups

| | Constipation | Treatment | Admin. | Dose/ | |
|---|---|---|---|---|---|
| Group # | Induction | Regimen | Route | Animal | |
| G1 | No | PBS | Intraperitoneal | — | |
| G2 | No | PEG-GALR2 | Intraperitoneal | 1 | mg/kg |
| G3 | No | PBS | Intranasal | — | |
| G4 | No | PEG-GALR2 | Intranasal | 10 | μg |
| G5 | No | bisacodyl | Oral | 100 | mg/kg |

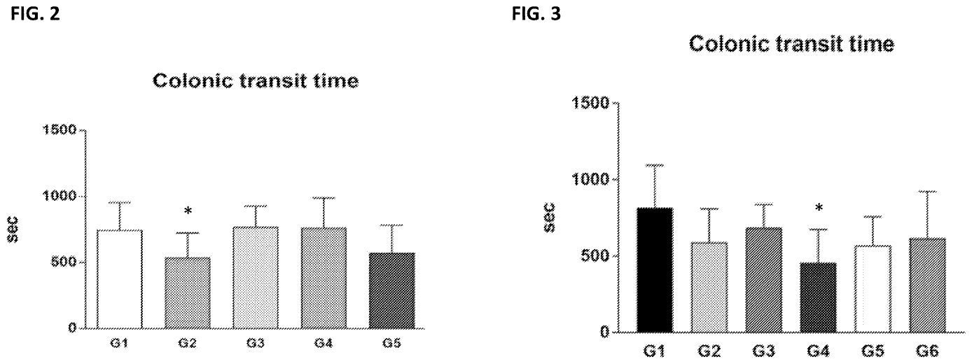

As shown in FIG. 2, animals that received intraperitoneal administrations of the PEG-GALR2 agonist (i.e., G2) exhibited significantly decreased colonic transit time compared to the control animals. However, in animals that received the PEG-GALR2 agonist intranasally (i.e., G4), the colonic transit time was generally comparable to the control animals.

To further assess the above-described effect on colonic transit time after intraperitoneal administration, varying doses (0.1 mg/kg, 0.3 mg/kg, or 1 mg/kg) of PEG-GALR2 agonist was again administered intraperitoneally to naïve mice as illustrated in FIG. 1 (i.e., daily for seven consecutive days). For comparison purposes, PEG-GALR2 agonist was administered to some of the animals subcutaneously (1 mg/kg). The different treatment groups are provided in Table 7 (below).

TABLE 7
Experimental Groups

| | Constipation | Treatment | Admin. | Dose/ | |
|---|---|---|---|---|---|
| Group # | Induction | Regimen | Route | Animal | |
| G1 | No | PBS | Intraperitoneal | — | |
| G2 | No | PEG-GALR2 | Intraperitoneal | 0.1 | mg/kg |
| G3 | No | PEG-GALR2 | Intraperitoneal | 0.3 | mg/kg |
| G4 | No | PEG-GALR2 | Intraperitoneal | 1 | mg/kg |
| G5 | No | PEG-GALR2 | Subcutaneous | 1 | mg/kg |
| G5 | No | bisacodyl | Oral | 100 | mg/kg |

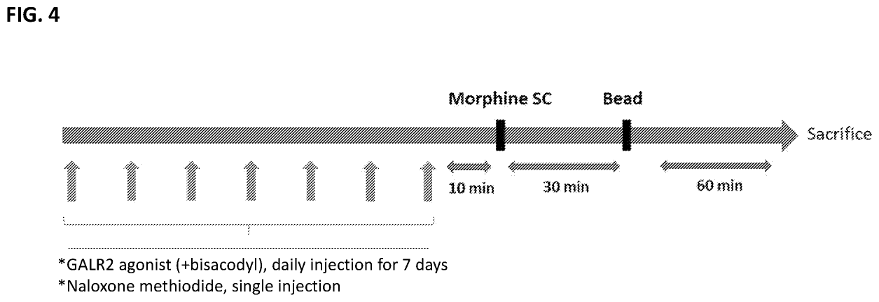

As shown in FIG. 3, the effect of GALR2 agonist on colonic transit time after intraperitoneal administration appeared to be dose dependent, with statistically significant decrease observed in animals treated with the GALR2 agonist at a dose of 1 mg/kg (i.e., G4). In animals that received the same dose (1 mg/kg) of GALR2 agonist subcutaneously, there was a decreased trend in colonic transit time compared to the negative control animals (compare G5 to G1).

The above results demonstrate the ability of the GALR2 agonists described herein to decrease colonic transit time.

### Example 2: Effect of GALR2 Agonist on Colonic Transit Time in Opioid-Induced Constipation (OIC) Mice

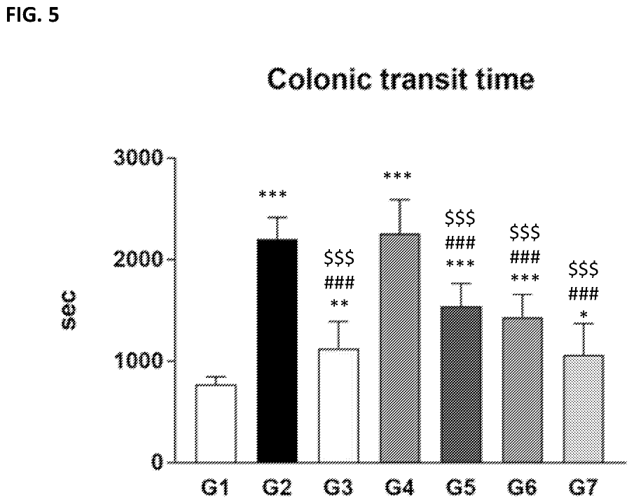

To assess whether the GALR2 agonists described herein could be useful in treating opioid-induced constipation, an OIC mouse model was used. As shown in FIG. 4, PEG-GALR2 agonist was administered daily to naïve mice for seven consecutive days. To further assess whether the route of administration has an effect, PEG-GALR2 agonist was administered to the mice intraperitoneally, intranasally, or subcutaneously. After the last administration, a dose of morphine (3 mg/kg) was subcutaneously administered to the animals to induce constipation (“OIC” mice). Then, a glass bead was rectally inserted, and colonic transit time was assessed by measuring the latency to glass bead expulsion by the animals. The following animals were used as controls: (i) normal naïve mice (no treatment and no morphine); (ii) OIC mice treated with vehicle control (negative control); (iii) OIC mice treated with bisacodyl (positive control); and (iv) OIC mice treated with naloxone methiodide (opioid receptor antagonist) (positive control). The different treatment groups are further described in Table 8 (below).

TABLE 8
Experimental Groups

| | Constipation | Treatment | Admin. | Dose / | |
|---|---|---|---|---|---|
| Group # | Induction | Regimen | Route | Animal | |
| G1 | No | — | — | — | |
| G2 | Yes | PBS | Intraperitoneal | — | |
| G3 | Yes | PEG-GALR2 | Intraperitoneal | 1 | mg/kg |
| G4 | Yes | PBS | Intranasal | — | |
| G5 | Yes | PEG-GALR2 | Intranasal | 10 | μg |
| G6 | Yes | Bisacodyl | Oral | 100 | mg/kg |
| G7 | Yes | Naloxone | Intraperitoneal | 10 | mg/kg |
| | | methiodide | | | |

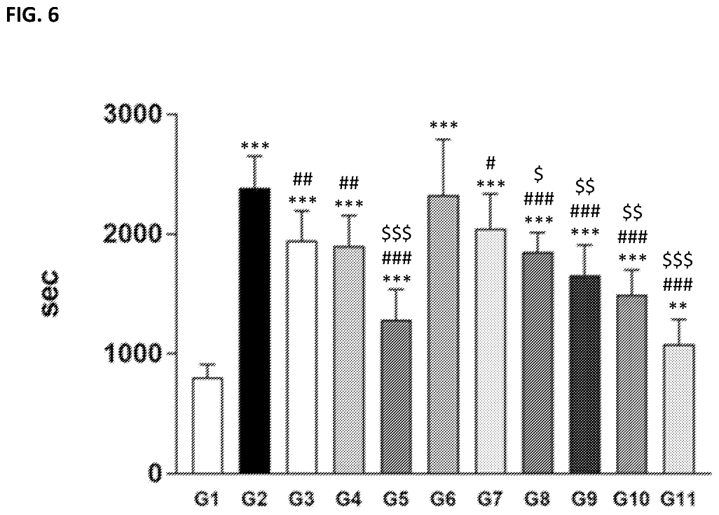

As shown in FIG. 5, both intraperitoneal and intranasal administration of the GALR2 agonist resulted in significant decrease in colonic transmit time in the OIC mice compared to the control animals. The decrease in colonic transit time in the intraperitoneally GALR2 agonist-treated animals was similar to that observed in the positive control animals (i.e., treated with naloxone methiodide). No significant differences were also observed between animals that received the GALR2 agonist intraperitoneally or subcutaneously.

The above results demonstrate that the GALR2 agonists described herein can also reduce colonic transit time in animals suffering from opioid-induced constipation. In contrast to that observed in naïve animals (see Example 1), administration route appeared to have minimal effect when the GALR2 agonist is administered in the presence of opioid-induced constipation.

### Example 3: Dose Dependent Effect of GALR2 Agonist after Intraperitoneal or Intranasal Administration in OIC Mice

To further characterize the effect that GALR2 agonists described herein has on opioid-induced constipation, OIC mouse model was used again. Briefly, as shown in FIG. 4, varying doses of PEG-GALR2 agonist was administered to mice for seven consecutive days. The PEG-GALR agonist was administered either intraperitoneally (0.1 mg/kg, 0.3 mg/kg, or 1 mg/kg) or intranasally (1 μg, 3 μg, or 10 μg). After the last administration, a dose of morphine (3 mg/kg) was subcutaneously administered to the animals to induce constipation (“OIC” mice). Then, a glass bead was rectally inserted, and colonic transit time was assessed by measuring the latency to glass bead expulsion by the animals. The following animals were used as controls: (i) normal naïve mice (no treatment and no morphine); (ii) OIC mice treated with vehicle control (negative control); (iii) OIC mice treated with bisacodyl (positive control); and (iv) OIC mice treated with naloxone methiodide (opioid receptor antagonist) (positive control). The different treatment groups are further described in Table 9 (below).

TABLE 9
Experimental Groups

| | Constipation | Treatment | Admin. | Dose/ | |
|---|---|---|---|---|---|
| Group # | Induction | Regimen | Route | Animal | |
| G1 | No | — | — | — | |
| G2 | Yes | PBS | Intraperitoneal | — | |
| G3 | Yes | PEG-GALR2 | Intraperitoneal | 0.1 | mg/kg |
| G4 | Yes | PEG-GALR2 | Intraperitoneal | 0.3 | mg/kg |
| G5 | Yes | PEG-GALR2 | Intraperitoneal | 1 | mg/kg |
| G6 | Yes | PBS | Intranasal | — | |
| G7 | Yes | PEG-GALR2 | Intranasal | 1 | μg |
| G8 | Yes | PEG-GALR2 | Intranasal | 3 | μg |
| G9 | Yes | PEG-GALR2 | Intranasal | 10 | μg |
| G10 | Yes | Bisacodyl | Oral | 100 | mg/kg |
| G11 | Yes | Naloxone | Intraperitoneal | 10 | mg/kg |
| | | methiodide | | | |

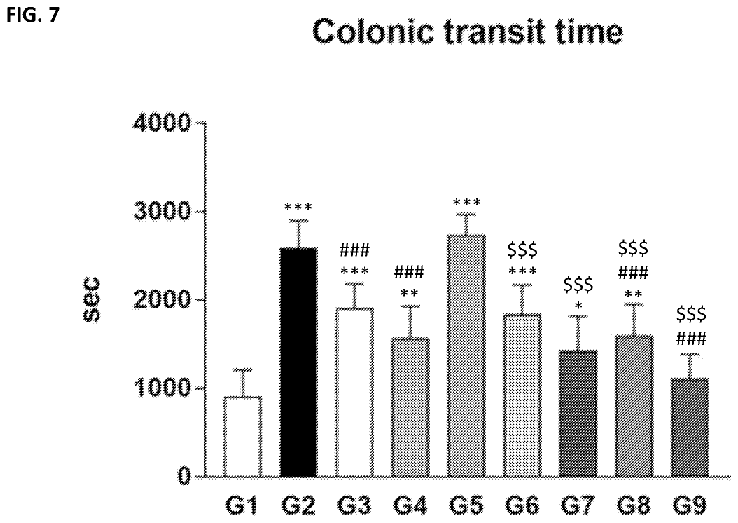

As shown in FIG. 6, and in agreement with the earlier data, all animals treated with a GALR2 agonist described herein had reduced colonic transit time as compared to the control animals. Regardless of the route of administration, the greatest decrease in colonic transit time was observed in animals that received the largest dose of the GALR2 agonist—i.e., 1 mg/kg for intraperitoneal administration and 10 μg for intranasal administration.

The above results demonstrate that the therapeutic effects observed with the GALR2 agonists described herein is dose dependent. The results further demonstrate that the administration route (at least between intraperitoneal and intranasal) likely has minimal impact on colonic transit time.

### Example 4: Dose Dependent Effect of GALR2 Agonist after Subcutaneous Administration in OIC Mice

To further characterize the effect that GALR2 agonists described herein has on opioid-induced constipation, OIC mouse model was used again. Briefly, PEG-GALR2 agonist (0.5 mg or 1 mg) was subcutaneously administered to mice for seven consecutive days. For comparison, the PEG-GALR2 agonist was administered intraperitoneally in some of the animals. After the last administration, a dose of morphine (3 mg/kg) was subcutaneously administered to the animals to induce constipation (“OIC” mice). Then, a glass bead was rectally inserted, and colonic transit time was assessed by measuring the latency to glass bead expulsion by the animals. The following animals were used as controls: (i) normal naïve mice (no treatment and no morphine); (ii) OIC mice treated with vehicle control (negative control); (iii) OIC mice treated with bisacodyl (positive control); and (iv) OIC mice treated with naloxone methiodide (opioid receptor antagonist) (positive control). The different treatment groups are further described in Table 10 (below).

TABLE 10
Experimental Groups

| | Constipation | Treatment | Admin. | Dose/ | |
|---|---|---|---|---|---|
| Group # | Induction | Regimen | Route | Animal | |
| G1 | No | — | — | — | |
| G2 | Yes | PBS | Intraperitoneal | — | |
| G3 | Yes | PEG-GALR2 | Intraperitoneal | 0.5 | mg/kg |
| G4 | Yes | PEG-GALR2 | Intraperitoneal | 1 | mg/kg |
| G5 | Yes | PBS | Subcutaneous | — | |
| G6 | Yes | PEG-GALR2 | Subcutaneous | 0.5 | mg/kg |
| G7 | Yes | PEG-GALR2 | Subcutaneous | 1 | mg/kg |
| G8 | Yes | Bisacodyl | Oral | 100 | mg/kg |
| G9 | Yes | Naloxone | Intraperitoneal | 10 | mg/kg |
| | | methiodide | | | |

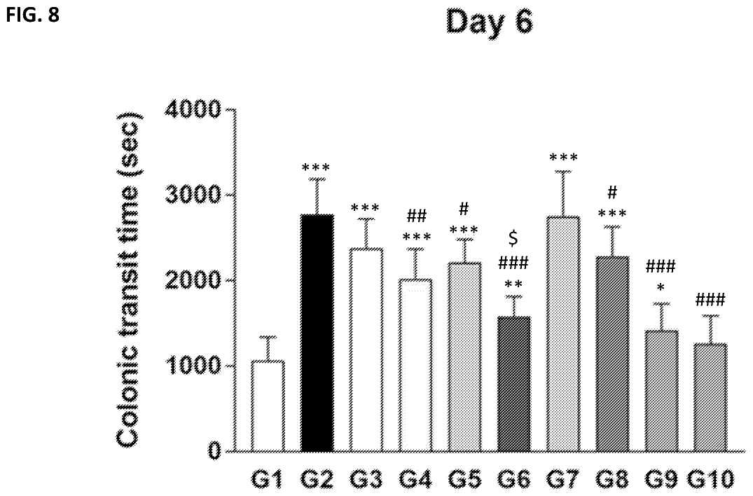

As shown in FIG. 7, again, all the animals that received the GALR2 agonist displayed reduced colonic transit time compared to PBS-treated animals. As observed earlier (Example 3), among animals that received the GALR2 agonist intraperitoneally, the greatest therapeutic effect was observed in mice that received the higher dose (i.e., 1 mg/kg), confirming the earlier described dose-effect. Similar dose effect was observed in mice that received the GALR2 agonist subcutaneously.

The above results further demonstrate the dose-dependent therapeutic effects of the GALR2 agonists described herein on colonic transit time. As in Example 3, the above results suggest that the particular route of administration may have minimal impact (at least between intraperitoneal and subcutaneous administration).

### Example 5: Comparison of Peg-GALR2, GALR2, and Wild-Type Spexin

To compare the therapeutic effects of the GALR2 agonists provided herein to wild-type spexin, OID mouse model was used again. Briefly, as shown in Table 11 (below), animals subcutaneously received one of the following treatments: daily for seven consecutive days (i) PEG-GALR2 (0.5 or 1 mg/kg); (ii) non-pegylated GALR2 (“GALR2”) (0.5 or 1 mg/kg); and (iii) wild-type spexin (0.5 or 1 mg/kg). Then, OIC was induced in the animals and colonic transit time was measured as described in the earlier examples (see, e.g., Example 2). The different treatment groups are further described in Table 11 (below).

TABLE 11
Experimental Groups

| | Constipation | Treatment | Admin. | Dose/ | |
|---|---|---|---|---|---|
| Group # | Induction | Regimen | Route | Animal | |
| G1 | No | — | — | — | |
| G2 | Yes | PBS | Subcutaneous | — | |
| G3 | Yes | PEG-GALR2 | Subcutaneous | 0.5 | mg/kg |
| G4 | Yes | PEG-GALR2 | Subcutaneous | 1 | mg/kg |
| G5 | Yes | GALR2 | Subcutaneous | 0.5 | mg/kg |
| G6 | Yes | GALR2 | Subcutaneous | 1 | mg/kg |
| G7 | Yes | WT Spexin | Subcutaneous | 0.5 | mg/kg |
| G8 | Yes | WT Spexin | Subcutaneous | 1 | mg/kg |
| G9 | Yes | Bisacodyl | Oral | 100 | mg/kg |
| G10 | Yes | Naloxone | Intraperitoneal | 10 | mg/kg |
| | | methiodide | | | |

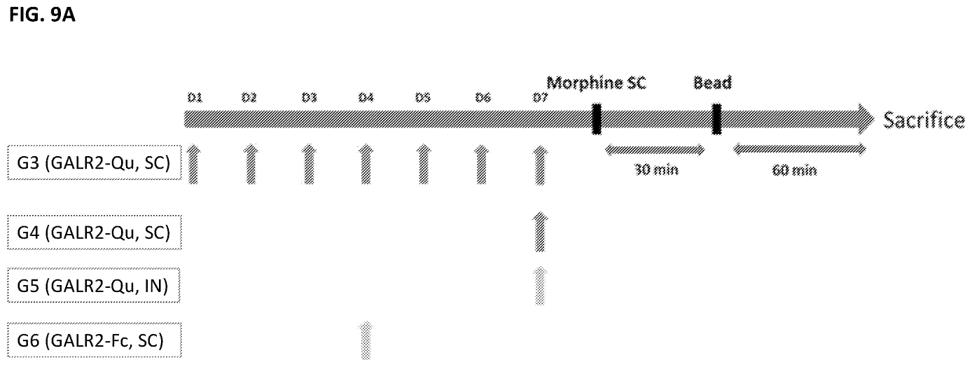

As shown in FIG. 8, in all treated animals (i.e., G3-G8), there was a dose dependent decrease in colonic transit time. As compared to the different treatment regimens, the therapeutic effect on colonic transit time was much more profound in GALR2 agonist-treated animals as compared to those animals treated with the wild-type spexin. No significant differences were observed between pegylated GALR2 and non-pegylated GALR2.

The above results confirm the superior therapeutic effects associated with the GALR2 agonists described herein. Compared to wild-type spexin (a natural ligand for GALR2), the GALR2 agonists of the present disclosure are much more effective in reducing colonic transit time in subject suffering from opioid induced constipation.

### Example 6: GALR2 Agonist Administration Schedule on Therapeutic Effects

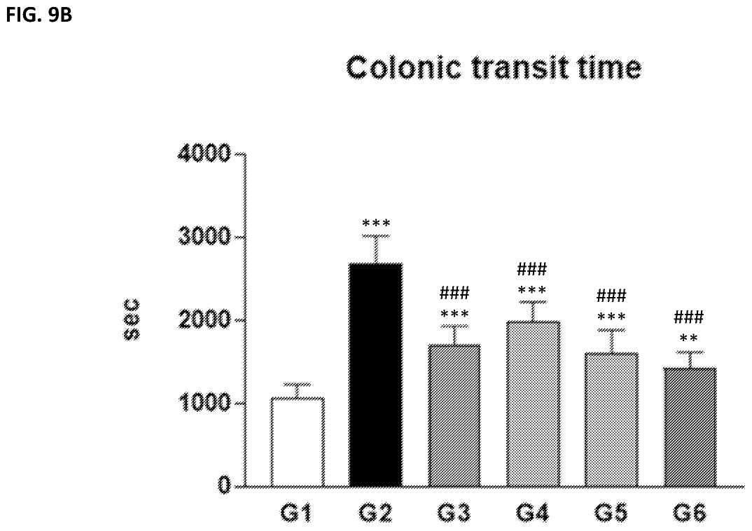

To assess whether the frequency of administration can be reduced without compromising therapeutic effects, a GALR2 agonist described herein was conjugated to Fc (GALR2-Fc). Then, mice received either the GALR2-Fc or the non-Fc conjugated GALR2 as further described in Table 12 (below) and illustrated in FIG. 9A. After the final administration, OIC was induced in the animals and colonic transit time was measured as described in the earlier examples (see, e.g., Example 2).

TABLE 12
Experimental Groups

| | OIC | | | | | |
|---|---|---|---|---|---|---|
| | Induc- | Treatment | Admin. | Fre- | | |
| Group # | tion | Regimen | Route | quency | Dose/Animal | |
| G1 | No | — | — | | — | |
| G2 | Yes | PBS | Subcutaneous | | — | |
| G3 | Yes | GALR2-Qu | Subcutaneous | Daily | 0.5 | mg/kg |
| | | | | for | | |
| | | | | 7 days | | |
| G4 | Yes | GALR2-Qu | Subcutaneous | Single | 1 | mg/kg |
| | | | | dose | | |
| G5 | Yes | GALR2-Qu | Intranasal | Single | 10 | μg/head |
| | | | | dose | | |
| G6 | Yes | GALR2-Fc | Subcutaneous | Single | 35 | mg/kg |
| | | | | dose | | |

As shown in FIG. 9B, a single subcutaneous administration of the GALR2 agonist described herein (i.e., G4) resulted in comparable reduction on colonic transit time, as compared to animals that received daily subcutaneous administration of the GALR2 agonist for seven total days (i.e., G3). Similar reduction was also observed in animals that received a single intranasal administration of the GALR2 agonist (i.e., G5). Compared to the control animals (i.e., G2), single subcutaneous administration of Fc-conjugated GALR2 also resulted in significant reduction in colonic transit time.

The above results suggest that a single administration of the GALR2 agonists described herein is capable of having therapeutic effects (e.g., reduced colonic transit time).

### Example 7: Effect of GALR2 Agonist Administration on Repetitive OIC Induction

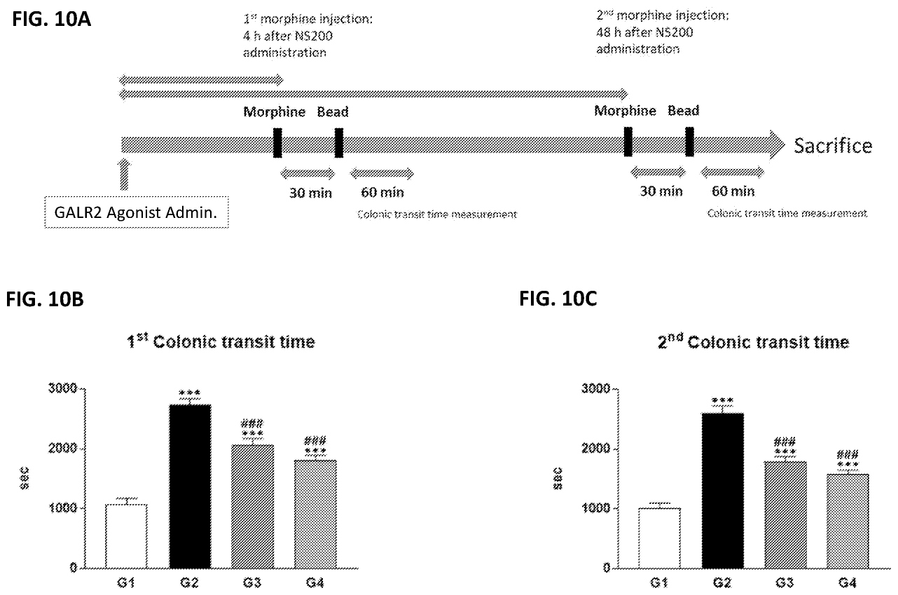

To assess the long-term therapeutic effects of GALR2 administration, the OIC mouse model was used. As illustrated in FIG. 10A, mice received a single administration (either subcutaneously or intranasally) of the GALR2 agonist. The following animals were used as controls: (i) normal naïve mice (no treatment and no morphine); and (ii) OIC mice treated with vehicle control (negative control). Then, OIC was induced and colonic transit time measured as described in the earlier examples (see, e.g., Example 2). After some time (about 44 hours), OIC was again induced in the animals and a second colonic transit time was measured in the animals.

In agreement with the results from the earlier examples (see, e.g., Example 5), single administration of the GALR2 agonist resulted in decreased colonic transit time compared to the control OIC mice (see FIG. 10B). The decrease was observed regardless of whether the GALR2 agonist was administered subcutaneously or intranasally. Similar reduction in colonic transit time was observed even after the second OIC induction (see FIG. 10C).

Collectively, the above results demonstrate that a single administration of the GALR2 agonists can have long-lasting significant effects on colonic transit time, further confirming the superior therapeutic potential of the GALR2 agonists described herein on treating various diseases, e.g., associated with constipation.

### Example 8: Effect of a 4th Amino Acid Position Substitution in GALR2 Agonist and Change in Solubility

To further characterize the GALR2 agonist provided herein, a solubility analysis was performed. Specifically, the following three GALR2 agonist peptides which differ at the 4th amino acid position were tested: (1) nWTaNAALYLFGPq (i.e., “D-alanine”; SEQ ID NO: 87), (2) NWTeNAALYLFGPq (i.e., “D-glutamic acid”; SEQ ID NO: 88), and (3) NWTrNAALYLFGPq (i.e., “D-arginine”; SEQ ID NO: 89). For each of the amino acid sequences provided, lowercase letter represents a D-form of the amino acid. D-glutamic acid and D-arginine peptides were further pegylated at the N-terminal end. All three GALR2 agonist peptides tested had an NH₂-group conjugated to the C-terminal end to block C-terminal degradation of the synthesized peptide and is not necessary for the functional activity of the peptide. The specific structure of the three GALR2 agonist peptides tested were as follows: (1) nWTaNAALYLFGPq-NH₂, (2) PEG2-NWTeNAALYLFGPq-NH₂, and (3) PEG2-NWTrNAALYLFGPq-NH₂.

Solubility of each of the GALR2 agonists peptides were assessed using two different solvents: Solvent 1: pH 5.5, L-histidine (10 mM) and Solvent 2: pH 6.0, phosphate buffer). The three different GALR2 agonist peptides were also evaluated under three different concentrations: 1.0 mg/100.0 μL, 1.0 mg/1.0 mL, and 0.1 mg/1.0 mL.

The peptides required for each of the corresponding concentrations above was added to the respective solvent and mixed via vigorously shaking for 30 seconds every 5 minutes at 20±5° C. The dissolution state of the peptide protein powder in the solvent was then checked approximately 30 minutes later.

TABLE 13
Experimental Groups

| 4^{th} Amino Acid | | Buffer 1 | Buffer 2 |
|---|---|---|---|
| Sequence Variation | Concentration | (pH 5.5) | (pH 6.0) |
| D-Alanine | 1.0 mg/100.0 μL | X | X |
| (SEQ ID NO: 87) | 1.0 mg/1.0 mL | X | X |
| | 0.1 mg/1.0 mL | X | X |
| D-Glutamic Acid | 1.0 mg/100.0 μL | X | X |
| (SEQ ID NO: 88) | 1.0 mg/1.0 mL | X | X |
| | 0.1 mg/1.0 mL | X | ◯ |
| D-Arginine | 1.0 mg/100.0 μL | X | X |
| (SEQ ID NO: 89) | 1.0 mg/1.0 mL | ◯ | X |
| | 0.1 mg/1.0 mL | ◯ | ◯ |

“◯” as shown above in the middle right column (Buffer 1) designates complete peptide solubilization as deteremined through visual inspection. “X” as shown above in the far right column (Buffer 2) designates the lack of peptide solubility through visual inspection.

As observed in Table 13 above, both the D-glutamic acid peptide (i.e., comprising a D-glutamic acid substitution at the 4^{th} amino acid position) and D-arginine peptide (i.e., comprising a D-arginine substitution at the 4^{th} amino acid position) exhibited improved solubility as compared to the D-alanine peptide (i.e., comprising a D-alanine substitution at the 4th amino acid position). Specifically, with the D-glutamic acid peptide, improved solubility was observed when dissolved in Solvent 2 at a concentration of 0.1 mg/1.0 mL. With the D-arginine peptide, improved solubility was observed when dissolved in Solvent 1 at a concentration of 0.1 mg/1.0 mL. Improved solubility was also observed for the D-arginine peptide in both Solvent 1 and Solvent 2 when dissolved at a concentration of 0.1 mg/1.0 mL.

Collectively, the results above demonstrate that certain amino acid substitutions (e.g., D-glutamic acid or D-arginine) at the 4th amino acid substitution can be useful in improving the solubility of a GALR2 agonist.

### Example 9: Effect of a 4th Amino Acid Position Substitution in GALR2 Agonist and In Vitro Potency

In addition to solubility (described above in Example 8), the in vitro potency of the three GALR2 agonist peptides described in Example 7 was next assessed. Specifically, mGqi-hGALR2-SRE Luc expressing cell lines were treated with the different peptides and SRE luciferase activity was measured (as a measure of intracellular activity mediated by the GALR2 agonist peptides).

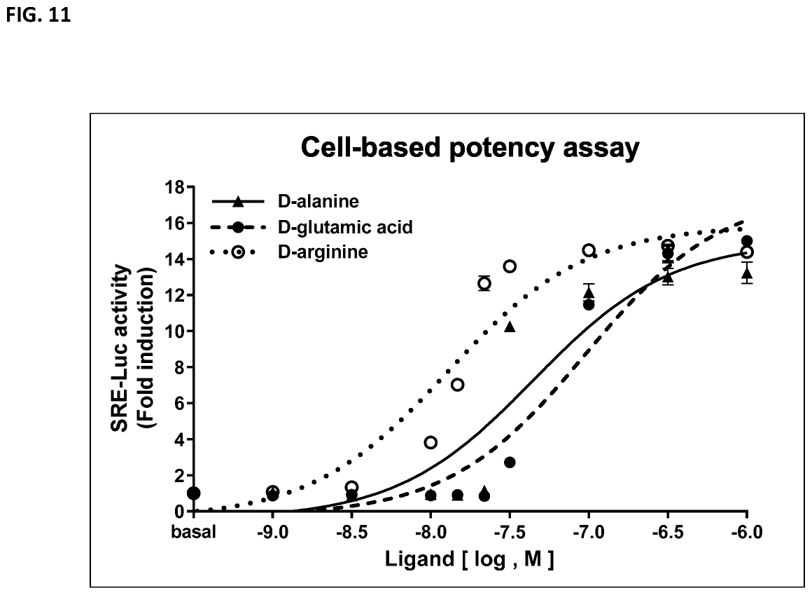

The Eₘₐₓ values of the three GALR2 agonist peptides were determined to be similar. Table 14 provides the log EC₅₀ (mean±SEM) values. Additionally, as shown in FIG. 11, the D-glutamic acid peptide was found to have a similar or lower EC₅₀ as compared to the D-alanine peptide. As for the D-arginine peptide, there was an approximately 3.5 fold increase in EC₅₀ value as compared to the D-alanine peptide.

TABLE 14
Potency

| 4^{th} Amino Acid | logEC50 (mean ± |
|---|---|
| Sequence Variation | SEM) |
| D-Alanine | −7.35 ± 0.15 |
| (SEQ ID NO: 87) | |
| D-Glutamic Acid | −7.02 ± 0.1 |
| (SEQ ID NO: 88) | |
| D-Arginine | −7.89 ± 0.1 |
| (SEQ ID NO: 89) | |

The above results further confirm the improved properties (e.g., potency) associated with GALR2 agonist peptides with certain amino acid substitutions (e.g., D-arginine) at the 4th amino acid substitution.

It is to be appreciated that the Detailed Description section, and not the Summary and Abstract sections, is intended to be used to interpret the claims. The Summary and Abstract sections can set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

What is claimed is:

1. A method of treating a gastrointestinal disorder in a subject in need thereof comprising administering to the subject a galanin receptor type 2 (GALR2) agonist, a nucleic acid encoding the GALR2 agonist, or a vector comprising the nucleic acid, wherein the GALR2 agonist comprises the amino acid sequence set forth in
X¹WX³X⁴X⁵X⁶X⁷X⁸YLX¹¹X¹²X¹³X¹⁴ (SEQ ID NO: 1), wherein:
X¹ is asparagine (N), glycine (G), pyroglutamate (pQ), or citrulline (Cit);
X³ is threonine (T), alanine (A), or lysine (K);
X⁴ is proline (P), leucine (L), glutamate (E), arginine (R), alanine (A), or valine (V);
X⁵ is asparagine (N) or glutamine (Q);
X⁶ is alanine (A) or serine(S);
X⁷ is alanine (A) or methionine (M);
X⁸ is leucine (L), glutamine (Q), or glycine (G);
X¹¹ is leucine (L), phenylalanine (F), tyrosine (Y), or aspartic acid (D);
X¹² is glycine (G) or alanine (A);
X¹³ is proline (P), arginine (R), or alanine (A);
X¹⁴ is glutamine (Q), histidine (H), or valine (V); and
wherein the GALR2 agonist specifically activates GALR2.

2. The method of claim 1, wherein the gastrointestinal disorder comprises a constipation, neurogenic bowel dysfunction (NBD), or both.

3. The method of claim 2, wherein the constipation comprises an opioid-induced constipation (OIC).

4. A method of regulating a bowel movement in a subject in need thereof comprising administering to the subject a galanin receptor type 2 (GALR2) agonist, a nucleic acid sequence encoding the GALR2 agonist, or a vector comprising the nucleic acid, wherein the GALR2 agonist comprises the amino acid sequence set forth in
X¹WX³X⁴X⁵X⁶X⁷X⁸YLX¹¹X¹²X¹³X¹⁴ (SEQ ID NO: 1), wherein:
X¹ is asparagine (N), glycine (G), pyroglutamate (pQ), or citrulline (Cit);
X³ is threonine (T), alanine (A), or lysine (K);
X⁴ is proline (P), leucine (L), glutamate (E), arginine (R), alanine (A), or valine (V);
X⁵ is asparagine (N) or glutamine (Q);
X⁶ is alanine (A) or serine(S);
X⁷ is alanine (A) or methionine (M);
X⁸ is leucine (L), glutamine (Q), or glycine (G);
X¹¹ is leucine (L), phenylalanine (F), tyrosine (Y), or aspartic acid (D);
X¹² is glycine (G) or alanine (A);
X¹³ is proline (P), arginine (R), or alanine (A);
X¹⁴ is glutamine (Q), histidine (H), or valine (V); and
wherein the GALR2 agonist specifically activates GALR2.

5. The method of claim 4, wherein regulating a bowel movement comprises regulating a colonic transit time in the subject.

6. The method of claim 5, wherein, after the administration of the GALR2 agonist, the colonic transit time in the subject is decreased compared to that of a reference subject (e.g., the subject prior to the administration and/or a corresponding subject who did not receive the administration).

7. The method of claim 6, wherein the colonic transit time is decreased by at least about 5%, at least about 10%, at least about 20%, at least about 30%, at least about 40%, at least about 50%, at least about 60%, at least about 70%, at least about 80%, at least about 90%, or about 100% as compared to that of the reference subject.

8. A method of treating an endocrine disorder in a subject in need thereof comprising administering to the subject a galanin receptor type 2 (GALR2) agonist, a nucleic acid sequence encoding the GALR2 agonist, or a vector comprising the nucleic acid sequence, which comprises the amino acid sequence set forth in
X¹WX³X⁴X⁵X⁶X⁷X⁸YLX¹¹X¹²X¹³X¹⁴ (SEQ ID NO: 1), wherein:
X¹ is asparagine (N), glycine (G), pyroglutamate (pQ), or citrulline (Cit);
X³ is threonine (T), alanine (A), or lysine (K);
X⁴ is proline (P), leucine (L), glutamate (E), arginine (R), alanine (A), or valine (V);
X⁵ is asparagine (N) or glutamine (Q);
X⁶ is alanine (A) or serine(S);
X⁷ is alanine (A) or methionine (M);
X⁸ is leucine (L), glutamine (Q), or glycine (G);
X¹¹ is leucine (L), phenylalanine (F), tyrosine (Y), or aspartic acid (D);
X¹² is glycine (G) or alanine (A);
X¹³ is proline (P), arginine (R), or alanine (A);
X¹⁴ is glutamine (Q), histidine (H), or valine (V); and
wherein the GALR2 agonist specifically activates GALR2.

9. The method of claim 4, wherein the endocrine disorder comprises a chronic renal failure, hypercalcemia, or both.

10. The method of any one of claims 1 to 9, wherein the N at X¹ is D-asparagine.

11. The method of any one of claims 1 to 10, wherein the W at position 2 of SEQ ID NO: 1 is D-tryptophan.

12. The method of any one of claims 1 to 11, wherein the A at X⁴ is D-alanine, D-glutamate, or D-arginine.

13. The method of any one of claims 1 to 11, wherein the V at X⁴ is D-valine.

14. The method of any one of claims 1 to 13, wherein the A at X⁶ is D-alanine.

15. The method of any one of claims 1 to 14, wherein the K at X¹¹ is D-lysine.

16. The method of any one of claims 1 to 14, wherein the A at X¹² is D-alanine.

17. The method of any one of claims 1 to 15, wherein the A at X¹³ is D-alanine.

18. The method of any one of claims 1 to 16, wherein the Q at X¹⁴ is D-glutamine.

19. The method of any one of claims 1 to 18, wherein the GALR2 agonist does not activate: (i) galanin receptor type 1 (GALR1), (ii) galanin receptor type 3 (GALR3), or (iii) both (i) and (ii).

20. The method of any one of claims 1 to 19, wherein X⁷ is A and X¹¹ is F.

21. The method of any one of claims 1 to 19, wherein X⁵ is N, X⁷ is A, and X¹¹ is F.

22. The method of any one of claims 1 to 19, wherein X⁵ is N, X⁷ is A, X¹¹ is F, and X¹³ is P.

23. The method of any one of claims 1 to 19, wherein the amino acid sequence of the GALR2 agonist comprises the sequence set forth in SEQ ID NO: 9, SEQ ID NO: 10, SEQ ID NO: 11, SEQ ID NO: 12, SEQ ID NO: 13, SEQ ID NO: 14, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 31, SEQ ID NO: 32, SEQ ID NO: 33, SEQ ID NO: 37, SEQ ID NO: 39, SEQ ID NO: 40, SEQ ID NO: 41, SEQ ID NO: 42, SEQ ID NO: 44, SEQ ID NO: 45, SEQ ID NO: 47, SEQ ID NO: 48, SEQ ID NO: 50, SEQ ID NO: 51; SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 87, SEQ ID NO: 88, SEQ ID NO: 89, SEQ ID NO: 90, or SEQ ID NO: 91.

24. The method of any one of claims 1 to 23, wherein the amino acid sequence of the GALR2 agonist is attached to a polyethylene glycol (PEG), an acetyl (Ac) group, or a Fmoc.

25. The method of claim 24, wherein X¹ is N, which is protected with the polyethylene glycol (PEG), acetyl (Ac) group, or Fmoc.

26. The method of any one of claims 1 to 24, wherein the amino acid sequence of the GALR2 agonist is attached to the NH₂ on the C-terminus.

27. The method of any one of claims 1 to 26, wherein the GALR2 agonist is administered to the subject intranasally, parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracistemally, intracapsularly, topically, orally, or combinations thereof.

28. The method of claim 27, wherein the GALR2 agonist is administered to the subject subcutaneously, intranasally, or intraperitoneally.

29. The method of any one of claims 1 to 28, wherein the GALR2 agonist is administered to the subject one time, two times, three times, four times, five times, six times, or seven times or more.

30. The method of any one of claims 1 to 29, further comprising administering an additional therapeutic agent to the subject.

31. The method of claim 30, wherein the additional therapeutic agent comprises a laxative (e.g., bisacodyl), opioid receptor antagonist (e.g., naloxone methiodide), irrigation (e.g., transanal or colonic), electrical stimulation, or combinations thereof.

32. The method of claim 30, wherein the additional therapeutic agent and the GALR2 agonist are administered to the subject concurrently.

33. The method of claim 31, wherein the additional therapeutic agent and the GALR2 agonist are administered to the subject sequentially.

34. The method of any one of claims 1 to 32, wherein the GALR2 agonist is administered to the subject as a freeze-dried powder or solution.


SEQUENCE LISTING (91 sequences)

SEQ ID NO: 1 - protein, 14 aa, synthetic construct
  VARIANT 1: note = Wherein the amino acid can be N, G, pQ, or Cit.
  VARIANT 3: note = wherein the Amino acid can be T, A, or K
  VARIANT 4: note = wherein the Amino acid can be P, L, E, R, A, or V.
  VARIANT 5: note = wherein the Amino acid can be N or Q.
  VARIANT 7: note = wherein the Amino acid can be A or M.
  VARIANT 6: note = wherein the Amino acid can be A or S.
  VARIANT 8: note = wherein the Amino acid can be L, Q, or G.
  VARIANT 11: note = wherein the Amino acid can be L, F, Y, or D.
  VARIANT 12: note = wherein the Amino acid can be G or A.
  VARIANT 13: note = wherein the Amino acid can be P, R, or A.
  VARIANT 14: note = wherein the Amino acid can be Q, H, or V.
NWXXXXXXYLXXXX

SEQ ID NO: 2-8 - skipped (7 entries)

SEQ ID NO: 9 - protein, 14 aa, synthetic construct
  source 1..14: note = [dN1]-GALR2 Agonist
  SITE 1: note = D-Asparagine
NWTPQAMLYLKGAQ

SEQ ID NO: 10 - protein, 14 aa, synthetic construct
  source 1..14: note = [pQ1]-GALR2 Agonist
  MOD_RES 1: note = pyroglutamate
QWTPQAMLYLKGAQ

SEQ ID NO: 11 - protein, 14 aa, synthetic construct
  source 1..14: note = [Ac-N1]-GALR2 Agonist
  MOD_RES 1: note = acetylation
NWTPQAMLYLKGAQ

SEQ ID NO: 12 - protein, 14 aa, synthetic construct
  source 1..14: note = [G1]-GALR2 Agonist
GWTPQAMLYLKGAQ

SEQ ID NO: 13 - protein, 14 aa, synthetic construct
  source 1..14: note = [dW2]-GALR2 Agonist
  SITE 2: note = D-Tryptophan
NWTPQAMLYLKGAQ

SEQ ID NO: 14 - protein, 14 aa, synthetic construct
  source 1..14: note = [2NaI2]-GALR2 Agonist
  MOD_RES 2: note = sodium (2Na)
NITPQAMLYLKGAQ

SEQ ID NO: 15 - skipped

SEQ ID NO: 16 - protein, 14 aa, synthetic construct
  source 1..14: note = [A3]-GALR2 Agonist
NWAPQAMLYLKGAQ

SEQ ID NO: 17 - protein, 14 aa, synthetic construct
  source 1..14: note = [K3]-GALR2 Agonist
NWKPQAMLYLKGAQ

SEQ ID NO: 18 - protein, 14 aa, synthetic construct
  source 1..14: note = [dA4]-GALR2 Agonist
  SITE 4: note = D-Alanine
NWTAQAMLYLKGAQ

SEQ ID NO: 19 - protein, 14 aa, synthetic construct
  source 1..14: note = [E4]-GALR2 Agonist
NWTEQAMLYLKGAQ

SEQ ID NO: 20 - protein, 14 aa, synthetic construct
  source 1..14: note = [L4]-GALR2 Agonist
NWTLQAMLYLKGAQ

SEQ ID NO: 21 - protein, 14 aa, synthetic construct
  source 1..14: note = [R4]-GALR2 Agonist
NWTRQAMLYLKGAQ

SEQ ID NO: 22 - protein, 14 aa, synthetic construct
  source 1..14: note = [dV4]-GALR2 Agonist
  SITE 4: note = D-Valine
NWTVQAMLYLKGAQ

SEQ ID NO: 23 - protein, 14 aa, synthetic construct
  source 1..14: note = [N5]-GALR2 Agonist
NWTPNAMLYLKGAQ

SEQ ID NO: 24 - protein, 14 aa, synthetic construct
  source 1..14: note = [dA6]-GALR2 Agonist
  SITE 6: note = D-Alanine
NWTPQAMLYLKGAQ

SEQ ID NO: 25 - protein, 14 aa, synthetic construct
  source 1..14: note = [A7]-GALR2 Agonist
NWTPQAALYLKGAQ

SEQ ID NO: 26 - protein, 14 aa, synthetic construct
  source 1..14: note = [G8]-GALR2 Agonist
NWTPQAMGYLKGAQ

SEQ ID NO: 27 - protein, 14 aa, synthetic construct
  source 1..14: note = [Q8]-GALR2 Agonist
NWTPQAMQYLKGAQ

SEQ ID NO: 28-30 - skipped (3 entries)

SEQ ID NO: 31 - protein, 14 aa, synthetic construct
  source 1..14: note = [F11]-GALR2 Agonist
NWTPQAMLYLFGAQ

SEQ ID NO: 32 - protein, 14 aa, synthetic construct
  source 1..14: note = [L11]-GALR2 Agonist
NWTPQAMLYLLGAQ

SEQ ID NO: 33 - protein, 14 aa, synthetic construct
  source 1..14: note = [Y11]-GALR2 Agonist
NWTPQAMLYLYGAQ

SEQ ID NO: 34-36 - skipped (3 entries)

SEQ ID NO: 37 - protein, 14 aa, synthetic construct
  source 1..14: note = [dA12]-GALR2 Agonist
  SITE 12: note = D-Alanine
NWTPQAMLYLKAAQ

SEQ ID NO: 38 - skipped

SEQ ID NO: 39 - protein, 14 aa, synthetic construct
  source 1..14: note = [P13]-GALR2 Agonist
NWTPQAMLYLKGPQ

SEQ ID NO: 40 - protein, 14 aa, synthetic construct
  source 1..14: note = [dQ14]-GALR2 Agonist
  SITE 14: note = D-Glutamine
NWTPQAMLYLKGAQ

SEQ ID NO: 41 - protein, 14 aa, synthetic construct
  source 1..14: note = [H14]-GALR2 Agonist
NWTPQAMLYLKGAH

SEQ ID NO: 42 - protein, 14 aa, synthetic construct
  source 1..14: note = [PEG]-GALR2 Agonist
  MOD_RES 1: note = PEGylation
NWTPQAMLYLKGAQ

SEQ ID NO: 43 - skipped

SEQ ID NO: 44 - protein, 14 aa, synthetic construct
  source 1..14: note = [Cit1]-GALR2 Agonist
  SITE 1: note = Citrulline
XWTPQAMLYLKGAQ

SEQ ID NO: 45 - protein, 13 aa, synthetic construct
  source 1..13: note = [Fmoc]-GALR2 Agonist
  MOD_RES 1: note = 9-fluorenylmethoxycarbonyl group
WTPQAMLYLKGAQ

SEQ ID NO: 46 - skipped

SEQ ID NO: 47 - protein, 20 aa, synthetic construct
  source 1..20: note = [SPX-M40]-GALR2 Agonist
NWTPQAMLYLKGAPPALALA

SEQ ID NO: 48 - protein, 14 aa, synthetic construct
  source 1..14: note = [A7F11]-GALR2 Agonist
NWTPQAALYLFGAQ

SEQ ID NO: 49 - skipped

SEQ ID NO: 50 - protein, 14 aa, synthetic construct
  source 1..14: note = [N5A7F11]-GALR2 Agonist
NWTPNAALYLFGAQ

SEQ ID NO: 51 - protein, 14 aa, synthetic construct
  source 1..14: note = [N5A7F11P13]-GALR2 Agonist
NWTPNAALYLFGPQ

SEQ ID NO: 52 - protein, 14 aa, synthetic construct
  source 1..14: note = [N5A7F11H14]-GALR2 Agonist
NWTPNAALYLFGAH

SEQ ID NO: 53 - protein, 14 aa, synthetic construct
  source 1..14: note = [PEG2]-GALR2 Agonist
  MOD_RES 1: note = PEGylation
NWTPNAALYLFGAH

SEQ ID NO: 54 - protein, 14 aa, synthetic construct
  source 1..14: note = [3-NO2-Y9]-GALR2 Agonist
  SITE 9: note = X=Y(NO2I)
NWTPQAMLXLKGAQ

SEQ ID NO: 55 - protein, 13 aa, synthetic construct
  source 1..13: note = [Fmoc-Qu]-GALR2 Agonist
  MOD_RES 1: note = 9-fluorenylmethoxycarbonyl group
WTPNAALYLFGPQ

SEQ ID NO: 56 - protein, 14 aa, synthetic construct
  source 1..14: note = [Fmoc-Qu-dQ14]-GALR2 Agonist
  SITE 14: note = D-Glutamine
  MOD_RES 1: note = 9-fluorenylmethoxycarbonyl group
NWTPNAALYLFGPQ

SEQ ID NO: 57 - protein, 14 aa, synthetic construct
  source 1..14: note = [Fmoc-Qu-dA4]-GALR2 Agonist
  MOD_RES 1: note = 9-fluorenylmethoxycarbonyl group
  SITE 4: note = D-Alanine
NWTANAALYLFGPQ

SEQ ID NO: 58 - protein, 14 aa, synthetic construct
  source 1..14: note = [Fmoc-Qu-dA4-dQ14]-GALR2 Agonist
  MOD_RES 1: note = 9-fluorenylmethoxycarbonyl group
  SITE 4: note = D-Alanine
  SITE 14: note = D-Glutamine
NWTANAALYLFGPQ

SEQ ID NO: 59 - protein, 14 aa, synthetic construct
  source 1..14: note = PEG-Qu-dA4-dQ14]-GALR2 Agonist
  MOD_RES 1: note = PEGylation
  SITE 4: note = D-Alanine
  SITE 14: note = D-Glutamine
NWTANAALYLFGPQ

SEQ ID NO: 60 - protein, 19 aa, synthetic construct
  source 1..19: note = Angiopep-2
  MOD_RES 19: note = hydroxyl group
TFFYGGSRGKRNNFKTEEY

SEQ ID NO: 61 - protein, 39 aa, synthetic construct
  source 1..39: note = ApoB (3371-3409)
SSVIDALQYKLEGTTRLTRKRGLKLATALSLSNKFVEGS

SEQ ID NO: 62 - protein, 18 aa, synthetic construct
  source 1..18: note = ApoE (159-167)2
LRKLRKRLLLRKLRKRLL

SEQ ID NO: 63 - protein, 8 aa, synthetic construct
  source 1..8: note = Peptide-22
  MOD_RES 1: note = acetyl group
  MOD_RES 8: note = amine group
  DISULFID 1..8: note = Interchain between cysteines
CMPRLRGC

SEQ ID NO: 64 - protein, 12 aa, synthetic construct
  source 1..12: note = THR
  MOD_RES 12: note = amine group
THRPPMWSPVWP

SEQ ID NO: 65 - protein, 12 aa, synthetic construct
  source 1..12: note = THR retro-enantio
  MOD_RES 12: note = amine group
PWVPSWMPPRHT

SEQ ID NO: 66 - protein, 9 aa, synthetic construct
  source 1..9: note = CRT
  DISULFID 1..9: note = Interchain between cysteines
CRTIGPSVC

SEQ ID NO: 67 - protein, 30 aa, synthetic construct
  source 1..30: note = Leptin30
YQQILTSMPSRNVIQISNDLENLRDLLHVL

SEQ ID NO: 68 - protein, 29 aa, synthetic construct
  source 1..29: note = RVG29
  MOD_RES 29: note = hydroxyl group
YTIWMPENPRPGTPCDIFTNSRGKRASNG

SEQ ID NO: 69 - protein, 16 aa, synthetic construct
  source 1..16: note = CDX
  MOD_RES 16: note = hydroxyl group
GREIRTGRAERWSEKF

SEQ ID NO: 70 - protein, 18 aa, synthetic construct
  source 1..18: note = Apamin
  DISULFID 1..11: note = Interchain between cysteines
  DISULFID 3..15: note = Interchain between cysteines
  MOD_RES 18: note = amine group
CNCKAPETALCARRCQQH

SEQ ID NO: 71 - protein, 9 aa, synthetic construct
  source 1..9: note = MiniAp-4
  MOD_RES 1: note = Dpr
  CROSSLNK 1..9: note = interchain between Diaminopropionic acid and Aspartic acid
XKAPETALD

SEQ ID NO: 72 - skipped

SEQ ID NO: 73 - protein, 13 aa, synthetic construct
  source 1..13: note = G23
HLNILSTLWKYRC

SEQ ID NO: 74 - protein, 7 aa, synthetic construct
  source 1..7: note = g7
  CARBOHYD 7: note = O-linked (GlcNH2)
GFTGFLS

SEQ ID NO: 75 - protein, 12 aa, synthetic construct
  source 1..12: note = TGN
TGNYKALHPHNG

SEQ ID NO: 76 - protein, 11 aa, synthetic construct
  source 1..11: note = TAT (47-57)
  MOD_RES 11: note = amine group
YGRKKRRQRRR

SEQ ID NO: 77 - protein, 18 aa, synthetic construct
  source 1..18: note = SynB1
RGGRLSYSRRRFSTSTGR

SEQ ID NO: 78 - skipped

SEQ ID NO: 79 - protein, 4 aa, synthetic construct
  source 1..4: note = PhPro
  MOD_RES 1..4: note = Phenylproline
  MOD_RES 4: note = amine group
PPPP

SEQ ID NO: 80 - protein, 349 aa, Homo sapiens
  source 1..349: note = GALR1 (UniProt: P47211-1)
MELAVGNLSEGNASWPEPPAPEPGPLFGIGVENFVTLVVFGLIFALGVLGNSLVITVLARSKPGKPRSTTNLFILNLSIADLAYLLFCIPFQATVYALPTWVLGAFICKFIHYFFTVSMLVSIFTLAAMSVDRYVAIVHSRRSSSLRVSRNALLGVGCIWALSIAMASPVAYHQGLFHPRASNQTFCWEQWPDPRHKKAYVVCTFVFGYLLPLLLICFCYAKVLNHLHKKLKNMSKKSEASKKKTAQTVLVVVVVFGISWLPHHIIHLWAEFGVFPLTPASFLFRITAHCLAYSNSSVNPIIYAFLSENFRKAYKQVFKCHIRKDSHLSDTKESKSRIDTPPSTNCTHV

SEQ ID NO: 81 - protein, 387 aa, Homo sapiens
  source 1..387: note = GALR2 (UniProt: O43603-1)
MNVSGCPGAGNASQAGGGGGWHPEAVIVPLLFALIFLVGTVGNTLVLAVLLRGGQAVSTTNLFILNLGVADLCFILCCVPFQATIYTLDGWVFGSLLCKAVHFLIFLTMHASSFTLAAVSLDRYLAIRYPLHSRELRTPRNALAAIGLIWGLSLLFSGPYLSYYRQSQLANLTVCHPAWSAPRRRAMDICTFVFSYLLPVLVLGLTYARTLRYLWRAVDPVAAGSGARRAKRKVTRMILIVAALFCLCWMPHHALILCVWFGQFPLTRATYALRILSHLVSYANSCVNPIVYALVSKHFRKGFRTICAGLLGRAPGRASGRVCAAARGTHSGSVLERESSDLLHMSEAAGALRPCPGASQPCILEPCPGPSWQGPKAGDSILTVDVA

SEQ ID NO: 82 - protein, 368 aa, Homo sapiens
  source 1..368: note = GALR3 (UniProt: O60755-1)
MADAQNISLDSPGSVGAVAVPVVFALIFLLGTVGNGLVLAVLLQPGPSAWQEPGSTTDLFILNLAVADLCFILCCVPFQATIYTLDAWLFGALVCKAVHLLIYLTMYASSFTLAAVSVDRYLAVRHPLRSRALRTPRNARAAVGLVWLLAALFSAPYLSYYGTVRYGALELCVPAWEDARRRALDVATFAAGYLLPVAVVSLAYGRTLRFLWAAVGPAGAAAAEARRRATGRAGRAMLAVAALYALCWGPHHALILCFWYGRFAFSPATYACRLASHCLAYANSCLNPLVYALASRHFRARFRRLWPCGRRRRHRARRALRRVRPASSGPPGCPGDARPSGRLLAGGGQGPEPREGPVHGGEAARGPE

SEQ ID NO: 83 - protein, 123 aa, Homo sapiens
  source 1..123: note = Galanin Precursor Peptide (UniProt: P22466-1)
MARGSALLLASLLLAAALSASAGLWSPAKEKRGWTLNSAGYLLGPHAVGNHRSFSDKNGLTSKRELRPEDDMKPGSFDRSIPENNIMRTIIEFLSFLHLKEAGALDRLLDLPAAASSEDIERS

SEQ ID NO: 84 - protein, 30 aa, Homo sapiens
  source 1..30: note = Galanin Mature Peptide
GWTLNSAGYLLGPHAVGNHRSFSDKNGLTS

SEQ ID NO: 85 - protein, 116 aa, Homo sapiens
  source 1..116: note = Human Spexin Precursor
MKGLRSLAATTLALFLVFVFLGNSSCAPQRLLERRNWTPQAMLYLKGAQGRRFISDQSRRKDLSDRPLPERRSPNPQLLTIPEAATILLASLQKSPEDEEKNFDQTRFLEDSLLNW

SEQ ID NO: 86 - protein, 14 aa, synthetic construct
  source 1..14: note = Spexin Mature Peptide
NWTPQAMLYLKGAQ

SEQ ID NO: 87 - protein, 14 aa, synthetic construct
  source 1..14: note = [dN1-dA4-dQ14]-GALR2 Agonist
  SITE 1: note = D-Asparagine
  SITE 4: note = D-Alanine
  SITE 14: note = D-Glutamine
  MOD_RES 14: note = amine group
NWTANAALYLFGPQ

SEQ ID NO: 88 - protein, 14 aa, synthetic construct
  source 1..14: note = [PEG-dE4-dQ14]-GALR2 Agonist
  MOD_RES 1: note = PEGylation
  SITE 4: note = D-Glutamate
  SITE 14: note = D-Glutamine
  MOD_RES 14: note = amine group
NWTENAALYLFGPQ

SEQ ID NO: 89 - protein, 14 aa, synthetic construct
  source 1..14: note = [PEG-dR4-dQ14]-GALR2 Agonist
  MOD_RES 1: note = PEGylation
  SITE 4: note = D-Arginine
  SITE 14: note = D-Glutamine
  MOD_RES 14: note = amine group
NWTRNAALYLFGPQ

SEQ ID NO: 90 - protein, 14 aa, synthetic construct
  source 1..14: note = [dE4-dQ14]-GALR2 Agonist
  SITE 4: note = D-Glutamate
  SITE 14: note = D-Glutamine
  MOD_RES 14: note = amine group
NWTENAALYLFGPQ

SEQ ID NO: 91 - protein, 14 aa, synthetic construct
  source 1..14: note = [dR4-dQ14]-GALR2 Agonist
  SITE 4: note = D-Arginine
  SITE 14: note = D-Glutamine
  MOD_RES 14: note = amine group
NWTRNAALYLFGPQ